(19) United States
(12) Patent Application Publication
(10) Pub. No.: US 2002/0160516 A1
(43) Pub. Date: Oct. 31, 2002

(54) Episomal expression vector for human gene therapy and expression system for production of proteins

(76) Inventor: Mark J. Cooper, Moreland Hills, OH (US)

Correspondence Address: BANNER & WITCOFF, 1001 G STREET N W, SUITE 1100, WASHINGTON, DC, 20001, US

(21) Appl. No.: 10/043,289

(22) Filed: Jan. 14, 2002

Related U.S. Application Data
(63) Continuation-in-part of application No. 09/935,368, filed on Aug. 24, 2001.
Application No. 09/935,368 is a continuation-in-part of application No. 09/473,646, filed on Dec. 28, 1999, now abandoned.
Application No. 09/473,646 is a 371 of international application No. PCT/US98/12777, filed on Jun. 19, 1998.
(63) Continuation-in-part of application No. 08/594,299, filed on Jan. 30, 1996, now Pat. No. 6,339,065.
Application No. 08/594,299 is a continuation of application No. 08/151,387, filed on Nov. 12, 1993, now abandoned.
(60) Provisional application No. 60/050,356, filed on Jun. 20, 1997.

(51) Int. Cl. C12N 15/86; C12N 7/01

(52) U.S. Cl. 435/456; 435/235.1; 435/320.1

(57) ABSTRACT

A method is described for expressing biologically functional proteins without host cell toxicity. This method takes advantage of the surprising replication activating ability of the 107/402-T antigen. The invention also provides expression vectors, human cells, and fusion proteins for practicing the method. The invention thus provides the art with an expression system for proteins which is useful to the pharmaceutical and biotechnology industries

This application is a continuation-in-part of co-pending application Ser. No. 09/935,368 filed Aug. 24, 2001, which is a continuation-in-part of Ser. No. 09/473,646 filed Dec. 28, 1999, now abandoned, which claims the benefit of PCT/US98/12777 filed Jun. 19, 1998, which claims the benefit of U.S. provisional application Ser. No. 60/050,356, filed Jun. 20, 1997. This application also is a continuation-in-part of co-pending application Ser. No. 08/594,299 filed Jan. 30, 1996, which is a continuation of Ser. No. 08/151,387 filed Nov. 12, 1993, now abandoned. Each of these applications is incorporated herein by reference.

The U.S. Government has a paid-up license in this invention and the right in limited circumstances to require the patent owner to license others on reasonable terms as provided for by the terms of IR55CA/OD66780, CA72737, and P30 CA43703 awarded by the National Institutes of Health.

### FIELD OF THE INVENTION

This invention is directed to papovavirus-derived episomes that replicate efficiently in mammalian cells, yielding stable transfectants having a high episomal copy number and expressing encoded genes at high levels. Papovavirus-derived episomes may be useful in gene therapy strategies to modulate the growth of bladder carcinoma cells.

The invention also relates to the area of protein expression. More particularly, the invention relates to human systems for expressing proteins of therapeutic value.

### BACKGROUND OF THE INVENTION

One approach to gene therapy of human cancer cells is to introduce vectors expressing antisense sequences to block expression of dominant oncogenes and growth factor receptors. However, high-level expression of the oncogenes requires comparable levels of antisense expression, which presents a considerable technical obstacle, particularly when using expression vectors having a limited potential for achieving multiple copies in stable transfectants. Human cells transduced by retroviral vectors have only one or several copies of integrated retrovirus in stable transfectants. In contrast, hundreds of copies of episomal plasmids can accumulate in stable transfectants because these vectors replicate extrachromosomally. One method to express high levels of antisense transcripts is to utilize episomal plasmid vectors than can replicate extrachromosomally in human cells.

Attempts to produce episomal vectors that will replicate in some types of human cells are reported by the literature. Episomal plasmids have been developed from several DNA viruses, including bovine papilloma virus (BPV) (Sarver et al., 1981, Mol. Cell. Biol., 1:486-496; DiMaio et al., 1982, Proc. Natl. Acad. Sci. USA.. 97:4030-4034), SV40 (Tsui et al., 1982, Cell, 30:499-508), Epstein-Barr virus (EBV) (Yates et al., 1985, Nature 313:812-815; Margolskee et al., 1988, Mol. Cell. Biol., 8:2837-2847; Belt et al., 1989, Gene 84:407-417; Chittenden et al., 1989, J. Virol. 63:3016-3025), and BK virus (BKV) (Milanesi et al., 1984, Mol. Cell. Biol. 4:1551-1560). Each of these episomal plasmids contains a viral origin of DNA replication and a virally encoded early gene that trans-activates the viral origin and allows the episome to replicate in the transfected host cell.

Although EBV-based episomes have been used to efficiently screen cDNA libraries, the EBV system has limited applications to non-lymphoid cell types (Vidal et al., 1990, Biochim. Biophys. Acta 1048:171-177)), and the EBV replicon is not active in many cell types. Additionally, EBNA-1 is one of several EBV latent genes that immortalize human lymphocytes, and transfection of the EBV-negative BJAB lymphoma cell line by EBNA-1 induces soft agar growth, indicating transformation of the cells. (Konoshita, 1990, Hokkaido Igaku Zasshi 65:362-375).

Furthermore, stable transfection efficiencies for EBNA-1 negative cell lines transduced by EBV episomal plasmids encoding EBNA-1 (transactivator) and ORI-P (EBV DNA origin) are low, not significantly better than non-episomal plasmids (Yates et al., 1985; Vidal et al., 1990. However, if EBNA-1 is expressed in cells prior to transfection, then a subsequent transfection with a plasmid containing ORI-P and a selectable marker can yield stable transfection efficiencies of up to 10% (Margolskee et al., 1988; Belt et al., 1989; Yates et al., 1984, Proc. Natl. Acad. Sci. USA 81:3806-3810; Lutfallia et al, 1989, Gene 76:27-39). Comparable results have been noted in a related system of COS cell clones expressing high levels of SV-T, which permit efficient replication of SV40 origin-containing plasmids in transient transfectants (Tsui et al., 1982; Rio et al., 1985, Science 227:23-28; Chittenden et al., 1991, J. Virol. 65:5944-5951).

In the COS cell system, however, episomal replication can proceed in a runaway fashion, resulting in up to 10⁴ episomal copies by 48 hours after transfection. Despite efficient episomal replication in transient transfectants, low stable transfection efficiencies have been noted in these studies (Chittenden et al., 1991; Roberts et al., 1986, Cell, 46:741-752). Presumably, most transient transfectants die secondary to episome-mediated cell death (Chittenden et al., 1991; Roberts et al., 1986).

However, transfection of COS cells by SV40 DNA origin-containing plasmids does produce stable transfectants having episomal plasmids (Tsui et al., 1982), and it may be possible to control runaway episomal replication by a variety of strategies, including use of replication control regions from other viruses. For example, runaway episomal replication in COS cell clones can be controlled by use of plasmids containing the SV40 DNA origin and regions of the bovine papilloma virus (BPV) replicon (Roberts et al., 1986; Roberts et al., 1988, Cell 52:397-404). These studies have identified two BPV sequences (NCOR I and NCOR II) that modulate runaway SV40 episomal replication in transient transfectants, and a third trans-suppressing factor encoded by 5′ sequences in the E1 open reading frame. Hybrid plasmids encoding the SV40 DNA origin and a 2113 bp EcoR1 fragment of BPV have substantially higher stable transfection efficiencies than pSV-NEO (Roberts et al., 1986). A DNA homology search failed to identify similar NCOR sequences in the BKV or SV40 replicon.

Thus, there remains a need for vectors which will replicate episomally in a controlled fashion in mammalian cells for gene therapy applications. In particular, there is a need for vectors that will replicate episomally in human cells without transforming the cells.

The production of large quantities of biologically functional therapeutic proteins requires an expression system which can both produce protein efficiently without toxic effects to the expression system itself and perform the required post-translational modifications. One approach to in vitro protein production is to transfect a bacterial or yeast cell with a plasmid encoding the protein of interest and culture the cell under conditions where the plasmid replicates to a high copy number, resulting in the potential for the production of large amounts of the desired protein. Due to differences in the biology of bacterial, yeast, and human cells, however, many non-human expression systems have very low efficiencies of producing functional product when the desired protein requires post-translational modification to be functional (Yarranton, 1990; Geisse et al. 1996). In mammalian cells, where post-translational modification of the desired protein may be accomplished more effectively, plasmids encoding the protein of interest are often replicated under control of a replication activator such as the SV40 large T antigen. Although the SV40 large T antigen is an efficient replication activator, high levels of extrachromosomal DNA replicating under the control of SV40 large T antigen normally are toxic to host cells (Gerard and Gluzman, 1985). This toxicity results in expression systems which function for only a short time.

Thus there is a need in the art for new systems for producing functional proteins for therapeutic uses.

### SUMMARY OF THE INVENTION

It is an object of the invention to provide a reagents and methods for production of a protein in a mammalian cell. This and other objects of the invention are provided by one or more of the embodiments described below.

One embodiment of the invention is a kit comprising a DNA molecule and a first episome. The DNA molecule comprises (1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen and (2) a first promoter which controls expression of the mutant form of the papovavirus large T antigen. The first episome comprises (1) the papovavirus origin of replication and (2) a coding sequence for a protein or a site for inserting the coding sequence for the protein.

Another embodiment of the invention is a kit comprising a mammalian cell, a DNA molecule, and a first episome. The DNA molecule comprises (1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen, wherein the DNA molecule is integrated into the genome of the mammalian cell and (2) a first promoter which controls expression of the mutant form of the papovavirus large T antigen. The first episome comprises (1) the papovavirus origin of replication, (2) a coding sequence for a protein or a site for inserting the coding sequence for the protein, and (3) a second promoter for controlling expression of the coding sequence for the protein.

Yet another embodiment of the invention is a kit comprising a mammalian cell and an episome. The episome comprises (1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen, (2) a promoter which controls expression of the mutant form of the papovavirus large T antigen, (3) the papovavirus origin of replication, and (4) a coding sequence for a protein or a site for inserting the coding sequence for the protein.

Still another embodiment of the invention is a mammalian cell comprising a DNA molecule and a first episome. The DNA molecule comprises (1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen and (2) a first promoter which controls expression of the mutant form of the papovavirus large T antigen. The first episome comprises (1) a coding sequence for a protein or a site for inserting the coding sequence for the protein and (2) the papovavirus origin of replication.

Yet another embodiment of the invention is a mammalian cell comprises a DNA molecule. The DNA molecule comprises (a) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen and (b) a promoter which controls expression of the mutant form of the papovavirus large T antigen.

A further embodiment of the invention is a method. A mammalian cell is cultured. The mammalian cell comprises a DNA molecule and a first episome. The DNA molecule comprises (1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen and (2) a first promoter which controls expression of the mutant form of the papovavirus large T antigen. The first episome comprises (1) a coding sequence for a protein and (2) the papovavirus origin of replication. The step of culturing is carried out under conditions suitable for expressing the protein.

Even another embodiment of the invention is a method. A mammalian cell is transfected with a first episome comprising (a) a coding sequence for a protein (b) a papovavirus origin of replication. The mammalian cell comprises a DNA molecule which comprises (a) a coding sequence for a mutant form of a papovavirus large T antigen which contains a replication-competent binding site for the papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen and (b) a first promoter which controls expression of the mutant form of the papovavirus large T antigen.

Still another embodiment of the invention is a kit comprising a mammalian cell, a first episome, and a second episome. The first episome comprises (1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen, wherein the DNA molecule is integrated into the genome of the mammalian cell and (2) a first promoter which controls expression of the mutant form of the papovavirus large T antigen. The second episome comprises (1) the papovavirus origin of replication, (2) a coding sequence for a protein or a site for inserting the coding sequence for the protein, and (3) a second promoter for controlling expression of the coding sequence for the protein.

Yet another embodiment of the invention is a method. A mammalian cell is transfected with an episome comprising (a) a coding sequence for a protein, (b) a papovavirus origin of replication, (c) a coding sequence for a mutant form of a papovavirus large T antigen which contains a replication-competent binding site for the papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen, and (d) a promoter which controls expression of the mutant form of the papovavirus large T antigen.

### BRIEF DESCRIPTION OF THE FIGURES

FIG. 1. Southern analysis of HT-1376 cells stably transfected with the BKV episomal vector pRP-cneoX. Cells were evaluated following 71 days of G418 selection. FIG. 1A. Hirt DNA from 3×10⁵ cells was loaded in lanes 1-4 and digested with restriction enzymes as indicated. Increasing amounts (50-400 pg) of BamHI-digested pRP-cneoX plasmid were loaded in lanes 5-9. FIG. 1B. Total cellular DNA (10 pg) from these same HT-1376/pRP-cneoX stable transfectants was digested with BamHI (lane 1). In lane 2 is 500 pg of BamHI-digested pRP-cneoX plasmid. Hybridization probe in both panels was ³²P-labelled pRP-cneoX.

FIG. 2. Expression of neomycin resistance gene mRNA in HT-1376 cells stably transfected with BKV episomal (pRP-cneoX lane 1) and non-episomal (pSV2NEO, lanes 2-4) expression vectors. FIG. 2A, hybridization probe, neomycin resistance gene. FIG. 2B, hybridization probe, β-actin.

FIG. 3. FIG. 3 shows that pRP-cneoX persists as an episomal plasmid in HT-1376 cells following withdrawal of selection pressure. Southern analysis of BamHI-digested Hirt supernatant DNA from 3×10⁵ HT-1376 pRP-cneoX transfectants grown in the presence (71 days, lane 1; 122 days, lane 2) or absence (16 days, lane 3; 34 days, lane 4; 47 days, lane 5; 64 days, lane 6) of G418. FIG. 3A, hybridization probe, ³²P-labeled pRP-cneoX. FIG. 3B, hybridization probe, 343 bp ³²P-labeled BamHI fragment of mouse mitochondrial DNA (ND1) obtained from pKSU1, a derivative of pAM1 (Martens et al. 1979, J. Mol. Biol., 135:327-351).

FIG. 4. FIG. 4 shows the persistence of neomycin resistance gene expression in HT-1376 pRP-cneoX transfectants following withdrawal of selection pressure. Northern blot analysis of 20 μg of RNA from HT-1376 transfectants in the presence (71 days, lane 1; 126 days, lane 6; 156 days, lane 7) or absence (16 days, lane 2; 34 days, lane 3; 47 days, lane 4; 64 days, lane 5) of G418. FIG. 4A, hybridization probe, ³²P-labeled BamHI-HindIII fragment of pSV2NEO containing coding sequences of the neomycin resistance gene. FIG. 4B, hybridization probe ³²P-labeled β-actin plasmid, pHFβA-1 (Gunning et al., 1983).

FIG. 5. FIG. 5 shows the location of point mutations in replication competent, transformation negative SV40 large T antigen (SV-T) mutants. The p53 and RB binding characteristics of 107/402-T, indicated in parenthesis, are predicted results.

FIG. 6. FIG. 6 shows Western blot analysis of single cell clones of 5637 cells stably transfected with SV-T or 107-T. Shown are clones of 5637 cells transfected with pRc/CMV.SV-T (lanes 1-3) or pRc/CMV.107-T (lanes 4-7). Blot was developed using anti-T antigen monoclonal antibody pAB 416 and a chemiluminescent development system (Amersham). 40 μg of lysate were loaded per lane.

FIG. 7. Southern blot analysis demonstrating that 107-T drives extrachromosomal replication of a plasmid (pSV2CAT containing the SV40 origin of DNA replication. 107-T 5637 clones C10 (no detectable expression) and E1 (high level expression) were transfected with pSV2CAT, and Hirt supernatant DNA was prepared approximately 4 days after transfection. Hirt supernatant DNA from approximately 5×10⁵ cells was loaded per lane, and evaluated before and after digested by DpnI, as indicated above. Hybridization probe was ³²P-labelled pSV2CAT.

FIG. 8. FIG. 8 demonstrates the presence of point mutations in codons 107 and 402 of replication-competent safety-modified SV40 large T antigen mutants.

FIGS. 9A-9B. FIGS. 9A-9B show co-immunoprecipitation analysis of binding of wild-type and mutant T antigens to human tumor suppressor gene products. In vitro translated T antigen (2×10⁵ dpm) was mixed with CV-1 extracts over producing human RB protein and anti-RB monoclonal antibody G3-245 (FIG. 9A, lanes 3-6), p53, and anti-p53 monoclonal antibody 1801 (FIG. 10A lanes 7-10), and p107 and anti-p107 monoclonal antibody SD9 (FIG. 9B, lanes 3-6). As controls, wild-type T antigen is immunoprecipitated with either anti-chromogranin A monoclonal antibody LKH210 (lane 1 of FIG. 9A and FIG. 9B) or anti-T antigen monoclonal antibody 416 (lane 2 of FIG. 9A and FIG. 9B).

FIGS. 10A-10B. FIGS. 11A-11B demonstrate that 107/402-T is replication-competent. FIG. 10A. HepG2 hepatoma cells were transfected with wild-type and mutant T antigen expression vectors, and total DNA was harvested 2 days post-transfection. DNA samples were sequentially digested with ApaI to linearize vector DNA and then with DpnI to distinguish amplified DNA from the input DNA used to transfect these cells. Since human cells lack adenine methylase activity, newly replicated DNA is resistant to DpnI digestion. Hence, the presence of unit length, linearized plasmid DNA (as indicated by the arrow) demonstrates newly replicated episome. Hybridization probe: pRC/CMV.107/402-T. FIG. 10B. Replication activity of wild-type SV40 large T antigen and SV40 T antigen mutants.

FIGS. 11A-11D. FIGS. 11A-11D show enhanced replication activity of 107/402-T in HepG2 cells. FIGS. 11A and 11B show FACS analysis of propidium iodide-stained HepG2 cells. FIG. 11C is a Southern blot which shows episomal copy number of wild-type and 107/402-T expression vectors in transfected HepG2 cells. FIG. 11D shows normalized replication activity of 107/402-T and wild-type T antigen expression vectors.

FIG. 12. FIG. 12 illustrates transgene (alkaline phosphatase) expression mediated by 107/402-T or wild-type T antigen in transiently transfected HepG2 cells.

FIG. 13. FIG. 13 depicts the time course of induction of 107/402-T expression vectors in an HT-1376 tet-off clone by removal of doxycycline.

FIG. 14. FIG. 14 shows dependence of 107/402-T expression on doxycycline concentration. Cells were harvested for Western blot analysis of T antigen expression 4 days after exposure to doxycycline.

FIG. 15. FIG. 15 shows the half-life of 107/402-T expression after addition of 3 ng/ml of doxycycline.

FIGS. 16A-16B. FIG. 16A shows cyclic production of secreted alkaline phosphatase (SEAP). FIG. 16B is a Western blot of protein extracts demonstrating 107/402-T antigen expression.

### DETAILED DESCRIPTION OF THE INVENTION

We have identified an episomal vector that efficiently replicates in transformed transitional epithelial cells (e.g., HT-1376 bladder carcinoma cell line). The vector (pRP-cneoX) contains a marker gene under control of the SV40 early promoter and a 3.2 kb segment of BKV which includes the BKV origin of replication and the BKV large T antigen under control of the BKV early promoter. Whereas the EBV episomal element was not active in HT-1376 transient transfectants, BKV episomes replicated extrachromosomally in these cells. More importantly, BKV episomes can replicate efficiently in HT-1376 cells without any apparent cellular toxicity, resulting in a high copy number of the episome in stable transfectants.

The copy number of BKV episome pRP-cneoX in HT-1376 cells stably transfected with this construct was approximately 150 copies per cell (see Example 1). This copy number compares to approximately 10-50 copies of EBV-derived episomes in lymphoblastoid cell lines and 10-80 copies of bovine papilloma virus-derived episomes in murine C127 cells (Sarver et al., 1981; DiMaio et al., 1982; Yates et al., 1985). The high copy number of pRP-cneoX in HT-1376 transfectants is likely responsible for the efficient vertical transfer of pRP-cneoX to the progeny of these HT-1376 transfectants over multiple generations. The soft agar cloning efficiencies of HT-1376 cells transfected with either integrating vector pSV2NEO or pRP-cneoX, and plated in the presence or absence of G418, were essentially identical. These data indicate that episomal transfer of the neomycin resistance gene to daughter cells was as efficient as when this gene is integrated into HT-1376 genomic DNA. This result was not unexpected, since the probability that a given daughter cell would not contain at least one copy of the episome would be very low assuming random partitioning of the large number of plasmid copies during cellular division.

Our data demonstrate that a BKV episomal expression vector can produce very high levels of transcription of a transfected gene in HT-1376 cells. There was approximately a 20-fold increase in the steady-state level of neomycin resistance gene expression in pRP-cneoX transfectants compared to transfectants which had 5 integrated copies of pSV2NEO. Since the neomycin resistance gene is transcriptionally regulated by the SV40 early promoter in both constructs, these data demonstrate that BKV episomal vectors can produce significantly higher levels of expression of a transfected gene that plasmid vectors that must integrate into the host cell genome to produce stable transfectants. This difference is presumably due in part to the higher copy number of pRP-cneoX (150 copies) compared to pSV2NEO (5 copies) in HT-1376 transfectants.

Comparison of Episomal Vectors

BKV-derived episomes have several properties that are distinct from EBV, BPV, and SV40-derived episomes. Despite the significant amino acid homology between the large T antigens from BKV and SV40 (Mann et al., 1984, Virol., 138:379-385), BKV episomes can yield stable, viable transfectants whereas SV40-based episomes replicate to such a high copy number that cell death typically ensues (Tsui et al., 1982; Roberts et al., 1986). This result may be due, in part, to differences in the level of T antigen present in these transfectants, characteristics of the DNA origins from these viruses, or presence of cis-regulatory sequences in the BKV episome that regulate DNA replication, as has been described in composite SV40-BPV-derived episomes (Roberts et al., 1986; Hambor et al., 1988, Proc. Natl. Acad. Sci. USA 85:4010-4104).

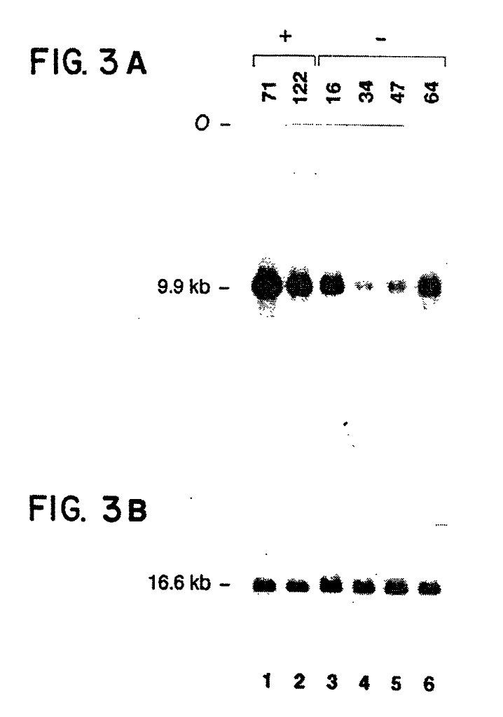

Significantly, BKV episomes appear to replicate once per cell cycle in stable transfectants, because the pRP-cneoX copy number reaches a stable plateau of approximately 150 copies per cell. Stable copy number is also characteristic of EBV and BPV-derived episomes, which can similarly yield viable, stable transfectants, albeit at lower copy number. In contrast to EBV-derived episomes (Yates et al., 1984; Hambor et al., 1988), however, the copy number of BKV episomes is maintained at unreduced levels after 2 months of growth in the absence of selection pressure. Fluctuations in pRP-cneoX copy number during the time course of G418 withdrawal (shown in FIG. 3) presumably represent a dynamic interplay between factors predisposed to maintain the presence of episomes (such as efficient episomal replication during the cell cycle and potential growth advantages present in cells expressing BKV large T antigen) and factors that may reduce episomal copy number (such as unequal partitioning of the episome during cell division, or destruction by cellular nucleases). Comparable to BKV episomes, BPV episomes can also be maintained at stable copy numbers in unselected, transformed C127 transfectants (Sarver et al., 1981; DiMaio et al., 1982). However, the higher copy number of BKV episomes in unselected transfectants is an advantage in strategies to utilize these episomes for gene therapy.

I. Definitions

In describing the present invention, the following terminology is used in accordance with the definitions kit out below.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., an intron-free coding sequence (cDNA) where the genomic coding sequence contains introns, or synthetic sequences having codons different than the native gene). Allelic variations or naturally occurring mutational events do not give rise to a heterologous region of DNA as defined herein.

A DNA “coding sequence” is a DNA sequence which is transcribed and translated into a polypeptide in vivo when placed under the control of appropriate regulatory sequences. A polyadenylation signal and transcription termination sequence will usually be located 3′ to the coding sequence. A “promoter” is a DNA regulatory region capable of binding RNA polymerase in a cell and initiating transcription of a downstream (3′ direction) coding sequence. A coding sequence under the control of a promoter in a cell is transcribed by RNA polymerase after the polymerase binds the promoter, the coding sequence being transcribed into mRNA which is then in turn translated into the protein encoded by the coding sequence.

“Transfection” of a cell occurs when exogenous DNA has been introduced inside the cell membrane. “Transformation” occurs when a cell population from primary cells or a cell line that only undergoes a finite number of divisions becomes immortalized, or when an immortal cell line acquires additional tumorigenic properties. Transformation can be detected by, for example, the ability of the transformed cell to form clones in soft agar or to form tumors in nude or SCID mice. A “clone” is a population of cells derived from a single cell or common ancestor by mitosis.

A “replicon” is any genetic element (e.g., plasmid, chromosome, virus) that functions as an autonomous unit of DNA replication in vivo.

A “vector” is a replicon, such as plasmid, phage or cosmid, to which another DNA segment (a heterologous segment) may be attached so as to bring about the replication of the attached segment.

An “episome” is a low molecular weight DNA molecule that resides in a cell separated from the cell's chromosome(s). Episomes replicate independently of mitotic replication of the chromosomes, being transmitted to daughter cells as part of the random reassortment of cellular contents during cell division. “Copy number” is the number of duplicate DNA molecules existing in an individual cell as episomes or is the number of duplicate sequences in the genome. Bacterial episomes are usually called plasmids.

“Foreign genes” are genes that are not found in the genome of the individual host cell. Foreign genes may be from the same species as the host or from different species. Where this invention describes transfection of a cell using DNA containing a foreign gene with the intent that the foreign gene will be expressed in the cell, the DNA will, of course, contain any control sequences necessary for expression of the foreign gene in the required orientation for expression.

Two DNA sequences that are substantially homologous can be identified by their ability to hybridize with each other in a Southern hybridization experiment, for example, under stringent conditions as defined for that particular system. Defining appropriate hybridization conditions is within the skill of the art. See, e.g., Maniatis et al., supra; DNA Cloning, vols. 1 and II supra; Nucleic Acid Hybridization, supra.

II. Description of the Vector

Papovaviruses

Papovaviruses are DNA viruses with double-stranded, covalently closed, circular genomes of approximately 5000 bp and icosahedral capsids containing three viral proteins. The papovaviruses infect a variety of hosts, including humans (BK virus and JC virus), monkeys (simian vacuolating virus (SV40) and lymphotropic papovavirus), baboon (simian agent 12), mouse (polyoma virus and K virus), hamster (hamster papovavirus), rabbit (rabbit kidney vacuolating virus), and budgerigar (budgerigar fledgling disease virus). These viruses have been judged to be related based on nucleotide sequence comparisons.

The viral genome is divided into early and late transcription regions, and contains a single origin of replication. Transcription begins from promoters near the origin of replication and proceeds bidirectionally—one direction for early transcripts and the other direction for late transcripts. The late transcriptional region encodes coat proteins (VP1, VP2, and VP3). The early transcriptional region encodes the T antigens, particularly the large T antigen which functions in viral DNA replication. The large T antigen also down-regulates early transcription by binding to viral DNA near the early promoter, activates cellular genes involved in DNA synthesis, and transforms primary cells in tissue culture.

Viral DNA replicates in the nucleus as “minichromosomes,” but viral DNA can replicate many times in a single cellular S phase. Viral DNA replication is initiated by the large T antigen, independently of its stimulation of cellular DNA synthesis. The large T antigen binds to viral DNA in the neighborhood of the origin of replication and unwinds the DNA helix, which is required for viral DNA replication. New viral DNA is then synthesized by cellular enzymes.

B. Episomal Amplification Cassette

To provide enhanced expression in gene therapy applications, episomal vectors must replicate extrachromosomally without transforming the transfected cell. This invention provides a replication cassette for such containing the essential elements of papovavirus replication. The replication cassette (or episomal amplification cassette) contains 1) a papovavirus origin of DNA replication (ORI); 2) a replication-competent, transformation-negative mutant form of the papovavirus large T antigen; and 3) a promoter to drive expression of the mutant T antigen. When the replication cassette of this invention is coupled with other DNA sequences in a circular DNA molecule, the DNA molecule will be replicated episomally by mammalian cells after transfection.

The initial BKV episomal vectors reported by Milanesi et al. (1984), contained a 3.2 kb fragment of BKV including the origin of DNA replication and the BKV large T antigen transcriptionally regulated by the BKV early promoter. As taught below and exemplified in the Examples, the BKV expression system may be modified according to this invention, so that it does not induce soft agar growth in nontumorigenic cells, yet retains the ability to replicate extrachromosomally. The components of the replication cassette will be selected according to the following criteria and assembled as described below.

1. Origin of Replication

The origin of replication in the replication cassette is selected from ORI sequences of one of the papovaviruses. DNA replication initiated at these loci is sensitive to control by the large T antigen of the same virus, and to a similar or lesser extent by large T antigen of other papovaviruses.

In the presence of a compatible large T antigen, the papovavirus origin will drive episomal replication. The origin/large T antigen combination should be tested to determine whether they drive replication of the episome. One simple test for replication competency is to transfect a population of cells which express the large T antigen mutant proposed for the replication cassette with a vector containing the proposed origin of replication and then monitor the transfected cells for synthesis of episomal DNA by Southern blot (see, e.g., Example 6C).

Particularly preferred is the BKV origin, which has been demonstrated to drive episomal replication with either BKV large T antigen (BK-T) or SV-T. Other preferred origins are those that drive replication in primates, including SV40, JC virus, lymphotropic papovavirus, and simian agent 12. Any papovavirus origin of replication that can be shown to drive episomal replication in human cells will be suitable for the replication cassettes of this invention.

The BKV replicon is active in the HT-1376 bladder carcinoma cell line, whereas the Epstein-Barr virus (EBV) replicon is not functional in these cells. BKV has a tropism for human uroepithelial cells (Arthur et al., 1986, N. Engl. J Med. 315:230-234), and an episomal vector derived from BKV will replicate efficiently in human bladder carcinoma cell lines. Hybrid SV40/BK virus-derived episomes replicated extrachromosomally in the nontumorigenic 5637 bladder cell line. These data suggest that the tissue tropism of viruses from which episomal constructs are derived may predict the cell type in which episomal constructs are active.

2. Large T Antigen Mutants

The replication activity of BKV episomes is dependent on expression of the BK-T. BK-T has a 75% amino acid homology to the SV40 large T antigen (SV-T) (Yang et al., 1979), a protein having well-described immortalization and tumorigenic properties (Shin et al., 1975, Proc. Natl. Acad. Sci. USA 72:4435-4439; Christian et al., 1987, Cancer Res. 47:6066-6073; Michalovitz et al., 1987, J. Virol. 61:2648-2654; Hanahan et al., 1989, Science 246:1265-1275; DeCaprio et al., 1988, Cell 54:275-283; Chen et al., 1990, J. Virol. 64:3350-3357; Chen et al., 1992, Oncogene 7:1167-1175). Similar to SV-T, BK-T can bind to and thereby inactivate wild-type p53 and retinoblastoma (RB) tumor suppressor gene products (Mann et al., 1984; Dyson et al., 1990, J. Virol. 64:1353-1356), the primary proposed mechanism by which these T antigens induce tumorigenic properties (DeCaprio et al., 1988; Chen et al., 1992). Transgenic mice expressing BK-T develop renal carcinomas and thymoproliferative disorders (Dalrymple et al., 1990, J. Virol. 64:1182-1191), and BK-T can transform NIH 3T3 cells and baby rat kidney cells (Nakshatri et al., 1988, J. Virol. 62:4613-4621). It is therefore possible that BKV episomal vectors containing wild-type BK-T could confer tumorigenic properties to some nontumorigenic cell lines, making such an episomal vector unsuitable for use in gene therapy, because the vector may be able to confer soft agar growth on cells in culture or induce neoplastic transformation in vivo.

However, the significant homology between SV-T and BK-T led to a specific strategy to solve this problem. SV-T can bind to the BKV origin of replication in vitro and can stimulate the replication of a plasmid containing the BKV origin of replication in COS cells (Ryder et al., 1983, Virol. 129:239-245; Deyerie et al., 1989, J. Virol. 63:356-365). Therefore, replication-competent SV-T mutants having suppressed transformation properties were examined as substitutes for BK-T to promote replication of BKV episomes without transformation.

Replication-competent, transformation-negative SV-T mutants

The domain of SV-T which binds the SV40 DNA origin is separate and distinct from the RB and p53 binding domains, as illustrated in Example 6 below. Three replication competent, transformation negative SV-T mutants are also illustrated. The first SV-T mutant is 107-T (also referred to as K1, Kalderon et al., 1984, Virol. 139:109-137), which is replication competent yet nontumorigenic in several cell types (DeCaprio et al., 1988; Chen et al., 1990; Chen et al., 1992; Kalderon et al., 1984; Cherington et al., 1988, Mol. Cell Biol. 8:1380-1384). 107-T differs from wild-type SV-T in a single base pair resulting in substitution of lysine for glutamic acid in codon 107. Codon 107 is in the RB binding domain of SV-T, and the inability of 107-T to bind RB most likely accounts for its nontumorigenic properties. The DNA binding region of 107-T is intact, however, and as shown below, we have determined that 107-T can drive replication of a test plasmid containing the SV40 DNA origin.

The second mutant is 402-T, which has a substitution of glutamic acid for asparagine in codon 402 (Lin et al., 1991, J. Virol. 65:2066-2072). The 402-T point mutation is in the p53 binding domain of SV-T, and 402-T fails to bind wild-type p53, although it appears to bind RB, and can also drive replication of the SV40 DNA origin. 402-T is nontransforming in human diploid fibroblast lines D.551 and WI-38 (Lin et al., 1991, J. Virol. 65:6447-6453).

Lastly, a novel SV-T mutant has been constructed which contains both point mutations found in 107-T and 402-T (107/402-T). This SV-T mutant will not bind either p53 or RB, and will have very low potential to confer tumorigenic properties. Replication competent 107/402-T will have particular value.

Integrating vectors encoding these three different SV-T mutants which have differing abilities to bind to wild-type p53 and RB have been prepared, and these SV-T mutant vectors have been transfected into nontumorigenic bladder cell lines. Single cell clones have been characterized which express the mutant SV-T molecules, yet remain nontumorigenic, and some of these clones have been shown to drive replication of plasmids containing SV40 DNA origins. This strategy has therefore been successful in modifying a papovavirus large T antigen for use in episomal vectors carrying an SV40 replicon for efficient expression of foreign genes in nontumorigenic cells.

The large T antigen mutants encoded by replication cassettes of this invention must be replication-competent and transformation-negative, that is they must induce DNA replication and not transform the host cell. Trans-activation of DNA replication can be tested using Southern blot analysis of Hirt supernatant or total cellular DNA extracted from transient episomal transfectants, as described above.

The transforming activity of the mutant large T antigen can be tested directly (see, e.g., Nakshatri et al. 1988) or cells transfected with an expression vector expressing the mutant T antigen can be tested for soft agar cloning activity or growth in nude or SCID mice to determine whether the mutant T antigen is transformation-negative. Alternatively, mutants may be selected based on negative binding studies with wild-type p53 and wild-type RB. One suitable assay measures binding by generating in vitro translated mutant large T antigen protein and mixing it with authentic wild-type p53 or RB (e.g., in vitro translated or baculovirus produced) before immunoprecipitation with antisera to p53 or RB, respectively, to immunoprecipitate these proteins and any T antigen proteins complexed to them. Western blots of the immunoprecipitate may be developed with antisera to large T antigen, which will detect mutant T antigens that are positive for binding.

An alternative procedure would be transfecting a population of mammalian cells expressing wild-type p53 and RB (preferably from a human cell line) with an expression vector so that the cells express the large T mutant (as detected by, e.g., binding to antisera for T antigen). The cells are then lysed and the lysate treated with antisera to p53 or RB. The immunoprecipitate is treated as before. This latter assay has some potential for false-negatives if, for instance, the amount of mutant T antigen expressed is significantly different from the amount of p53 or RB present, or if there are subtle mutations in the p53 or RB expressed by the test cell, but it more closely approximates the in vivo conditions.

A particularly preferred mutant large T antigen is the SV-T mutant 107/402-T described above. Other mutants of SV-T and other papovavirus large T antigens have been described in the literature, and additional mutants can be generated by well-known recombinant DNA techniques. These mutants will be suitable for the replication cassette of this invention, so long as they are replication-competent and transformation-negative as determined by the above tests.

3. Promoters

In general, replication-competent, transformation-negative papovavirus large T antigen will be transcriptionally regulated by either heterologous or homologous promoters. The heterologous promoters are usually promoters which are active in mammalian cells, such as mammalian promoters and mammalian viral promoters. Where the episomal amplification cassette is part of an episomal expression vector for gene therapy application, the promoter will, of course, be chosen to be active in the cell which is the target for expression of the foreign gene.

Some heterologous promoters, such as CMV immediate early promoter-enhancer, are not down-regulated by T antigen, thereby maximizing T antigen expression and consequently, episomal replication. This may be particularly advantageous in transient transfection strategies for gene therapy applications in which high level gene expression is desirable. Alternatively, use of homologous papovaviruses promoters, which are down regulated by T antigen, may constrain runaway episomal replication, thereby achieving controlled, stable expression. Such a promoter/T antigen/origin combination will provide high copy number, stable episomes in transfected cells.

Alternatively, the promoter controlling expression of the mutant large T antigen may be selected to regulate episomal replication. For example an inducible promoter (such as the metallothionene promoter) may be used, and replication of the episome will be amplified in the presence of the inducer. Alternatively, a promoter for a developmentally-controlled or tissue-specific gene (e.g., the breast specific promoter for the whey acidic protein gene, Shoeneberger et al., 1988, EMBO J. 7: 169-175) may be used to limit the amplification of the episome copy number to certain cell types where that promoter is active. In gene therapy using an episome which carries a foreign gene whose expression level is proportional to copy number, selection of the promoter controlling T antigen expression provides a measure of therapeutic control of expression.

4. Vectors for Insertion of Cassettes

Broadly, the vectors into which the replication cassette of this invention may be inserted may be any vector that will carry the cassette, and any associated foreign genes, into mammalian cells in which the particular papovavirus origin and large T antigen will drive replication of the vector. The vector, of course, will not contain any sequences that prevent replication from the papovavirus origin of replication in mammalian cells or prevent expression of any foreign gene inserted into the vector for gene therapy applications. Suitable vectors include bacterial plasmids, which are useful as shuttle vectors to produce large quantities of the vector containing the replication cassette in bacterial culture for subsequent use in transfection of mammalian cells. Other suitable vectors include well-known mammalian vectors, usually of viral origin, which are known to transfect mammalian cells, and are non-pathogenic, or of limited pathogenicity, including defective or mutant viruses (see, e.g., Hock et al. 1986, Nature 320:275-277; Sorrentino et al. 1992, Science 257:99-103; Bayle et al. 1993, Human Gene Therapy 4:161-170; Le Gal La Salle et al. 1993, Science 259:988-990; Quantin et al. 1992, Proc. Natl. Acad. Sci. USA 89:2581-2584; Rosenfeld et al. 1992, Cell 68:143-155). Where the vector is a mammalian virus, it is of course important that insertion of foreign genes into the viral genome does not destroy viral infectivity. Selection of a particular vector will take into account the particular mammal and the particular cell type in which episomal amplification is desired, and the skilled worker can readily select suitable vectors from among many available in art. (See, e.g., Sambrook et al., 1989, “Molecular Cloning: A Laboratory Manual”; Miller et al., 1989, BioTechniques 7:980-990; Salmons et al., 1993, Human Gene Therapy 4:129-141; Stratford-Perricaudet et al., 1991, in “Human Gene Transfer,” Cohen-Haguenauer et al., eds., John Libbery Eurotest Ltd. 219:51-61).

III. Method of Constructing the Vector

A. Sources of Component DNA Sequences

The DNA sequences of various papovaviruses are described in the literature, including the DNA sequences encoding the origin of replication, the early promoter, and the large T antigen. (See, e.g., (for SV40) Subramanian et al. 1977, J. Biol. Chem. 252:355-367; Reddy et al. 1978, Science 200:494-502; Fiers et al. 1978, Nature 273:113-120; Van Heuverswyn et al. 1978, Eur. J Biochem. 100:51-60; (for BKV) Yang et al. 1979, Science, 206:456-461; Deyerle et al. 1989, J. Virol. 63:356-365; (for hamster papovavirus) Delmas et al. 1985, EMBO J. 4:1279-1286; (for JC virus) Frisque et al. 1984, J. Virol. 51:458-469; (for polyoma) Zhu et al. 1984, J Virol. 51:170-180.) Clones containing many of the sequences are contained in various mammalian vectors available from commercial suppliers, such as Stratagene, Gibco-BRL Life Technologies, United States Biochemicals, and Promega. Clones containing the complete genomic sequence for BK virus, JC virus, K virus, polyoma virus, and SV40 are available from American Type Culture Collection, 12301 Parklawn Drive, Rockville, Md. 20852, U.S.A. (ATCC). Clones containing promoters, bacterial origins of replication, and a variety of vectors are also available from the commercial sources listed above or ATCC, as well as other sources well known to those skilled in the art of recombinant DNA manipulation. Specific sequences encoding particular proteins or regulatory sequences may be obtained from these clones using standard recombinant DNA techniques, such as those described below. The particular foreign genes whose expression in mammalian cells is desired, and sources for sequences encoding them, will be readily apparent to those skilled in the art of gene therapy.

B. Recombinant Procedures for Vector Construction

The practice of the present invention employs, unless otherwise indicated, conventional molecular biology, microbiology, and recombinant DNA techniques within the skill of the art. Such techniques are well known to the skilled worker and are explained fully in the literature. See, e.g., Maniatis, Fritsch & Sambrook, “Molecular Cloning: A Laboratory Manual” (1982); “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986); B. Perbal, “A Practical Guide to Molecular Cloning” (1984), and Sambrook et al., “Molecular Cloning: a Laboratory Manual” (1989).

DNA segments corresponding to the papovavirus origin of replication, the papovavirus large T antigen coding sequence and the papovavirus early promoter may be obtained from readily available recombinant DNA materials, such as those available from the ATCC, which include BK virus, JC virus, K virus, polyoma virus, and SV40 virus. DNA segments or oligonucleotides having specific sequences can be synthesized chemically or isolated by one of several approaches. The basic strategies for identifying, amplifying and isolating desired DNA sequences as well as assembling them into larger DNA molecules containing the desired sequence domains in the desired order, are well known to those of ordinary skill in the art. See, e.g., Sambrook et al., (1989); B. Perbal, (1984). Preferably, DNA segments corresponding to the papovavirus origin, large T antigen and early promoter may be isolated individually using the polymerase chain reaction (M. A. Innis et al., “PCR Protocols: A Guide To Methods and Applications,” Academic Press, 1990). A complete sequence may be assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311.

The assembled sequence can be cloned into any suitable vector or replicon and maintained there in a composition which is substantially free of vectors that do not contain the assembled sequence. This provides a reservoir of the assembled sequence, and segments or the entire sequence can be extracted from the reservoir by excising from DNA in the reservoir material with restriction enzymes or by PCR amplification. Numerous cloning vectors are known to those of skill in the art, and the selection of an appropriate cloning vector is a matter of choice (see, e.g., Sambrook et al., incorporated herein by reference). The construction of vectors containing desired DNA segments linked by appropriate DNA sequences is accomplished by techniques similar to those used to construct the segments. These vectors may be constructed to contain additional DNA segments, such as those encoding foreign genes for gene therapy, bacterial origins of replication to make shuttle vectors (for shuttling between prokaryotic intermediate hosts and mammalian final hosts), etc.

Procedures for construction and expression of mutant proteins of defined sequence are well known in the art. A DNA sequence encoding a known mutant of papovavirus large T antigen can be synthesized chemically or prepared from the wild-type sequence by one of several approaches, including primer extension, linker insertion and PCR (see, e.g., Sambrook et al.). Alternatively, additional mutants can be prepared by these techniques having additions, deletions and substitutions in the wild-type sequence. In either case, it is preferable to test the mutants to confirm that they are replication-competent and transformation-negative, by the assays described above. Mutant large T antigen protein for testing may be prepared by placing the coding sequence for the polypeptide in a vector under the control of a promoter, so that the DNA sequence is transcribed into RNA and translated into protein in a host cell transformed by this (expression) vector. The mutant large T antigen protein may be produced by growing host cells transfected by an expression vector containing the coding sequence for the mutant T antigen under conditions whereby the polypeptide is expressed. The selection of the appropriate growth conditions is within the skill of the art.

C. Intermediate Stage Vectors

Preferably the vector containing the replication cassette will also contain a functional bacterial origin of replication and selection markers that function in bacteria (i.e., a shuttle vector). These will allow cloning of the vector in bacteria to provide a stable reservoir of the vector for storage and to facilitate amplification, where large quantities of the vector containing the replication cassette and any associated foreign genes can be recovered from bacterial culture. The procedures, as well as appropriate bacterial origins and selection markers are well known in the art (see, e.g., Sambrook et al.). Alternatively, mammalian viral vectors may be amplified in mammalian cell culture, using well known techniques. Appropriate procedures for storage and standardization of preparations containing virus vectors or bacterial cells harboring shuttle plasmid vectors will be readily apparent to those skilled in the art.

D. Functional Tests of the Vector

Vectors containing the replication cassette of this invention will routinely be tested after they have been constructed to confirm that the vector is replication-competent and non-transforming. These tests will assure that sequences included in the vector do not interfere with the functioning of the replication cassette. Replication competence (i.e., that both the mutant large T antigen and the origin of replication are functional) is usually tested by transfecting a population of non-transformed cells of the target cell type with the vector and monitoring episomal DNA production by Southern blot. Stable transfectants from the replication test can be further tested for soft agar cloning activity or tumorigenesis in nude or SCID mice to confirm that the vector has not transformed the cells. Southern blots of DNA from the stable transfectants may be used to indicate whether they have integrated the vector into genomic DNA or if the vector is being carried as a stable episome.

IV. Use of the Vector

A. Therapeutic Use

Vectors for use in gene therapy are constructed by inserting the replication cassette of this invention into a suitable mammalian vector along with the foreign gene whose expression is desired, using standard recombinant DNA techniques as described above to produce an episomal expression vector. Cells are then transfected with these episomal expression vectors under conditions that maintain cell viability and the vectors replicate episomally in the cells. The episomal expression vectors may be administered to patients in a variety of ways.

In one embodiment cells are transfected with the episomal expression vector in vitro. Usually, appropriate cells are obtained from the patient, for instance peripheral blood monocytes from a blood sample, and these cells are transfected with the episomal expression vector before being re-introduced into the patient. Alternatively, stem cells in a population of the patient's cells are cultured to provide a large cell population, compatible with the patient, and the cells are transfected with the episomal expression vector in culture. Then the transfected cell population is re-introduced into the patient.

In another embodiment, the episomal expression vector is based on a mammalian virus which infects the patient mammal, containing a replication cassette according to this invention and a foreign gene. The viral episomal expression vector is administered to the patient, where it infects the patient's cells, and the episomal expression vector then replicates episomally in the cells.

In another embodiment, the episomal expression vector is introduced into the patient mammal in conjunction with liposomal or receptor-mediated delivery systems (see Felgner et al., 1987, Proc. Natl. Acad. Sci USA 84:7413-7417 and Zhu et al., 1993, Science 261:209-211, incorporated herein by reference). Once the patients' cells are transfected in vivo, the episomal expression vector will replicate extrachromasomally.

Expression of the foreign gene may occur once the cell has been transfected by the episomal expression vector. Usually the foreign gene will be expressed constitutively, and the level of expression will be controlled by the copy number of the episome. In another embodiment, expression of the foreign gene will be under control of a promoter than can be up- or down-regulated in a manner described above for expression of the mutant large T antigen. Selection of suitable promoters for control of the foreign gene will be apparent to the skilled worker, based on the desired clinical result.

While any gene that can be expressed in a mammalian cell may be incorporated into a transfection vector as the foreign gene according to this invention, preferred genes will be those whose expression in a target cell population will counter-act a disease process. For example, an episomal gene therapy vector could be used to target the immune system to kill cancer cells in vivo. Tumor cell lines transfected with cytokine cDNA have been successfully used as cancer vaccines (Connor et al., 1993, J. Exp. Med. 177:1127-1134; Golumbek et al., 1991, Science 254:713; Porgador et al., 1992, Cancer Res. 52:3678; Aoki et al., 1992, Proc. Natl. Acad. Sci. USA 89:3850) and transfection of tumor cells in vivo with appropriate episomal vectors will enhance tumor kill, since episomal replication in the tumor cell will efficiently produce the desired high local concentration of cytokines, thereby stimulating immune effector cells. One such example is introduction of episomal expression vectors encoding interleukin-2 into bladder cancer cells in vivo via instillation of liposome/DNA complexes directly into the bladder lumen. Another example is transfection of lung cancer cells in vivo with interleukin-6 via inhalation of aerosolized liposome/DNA complexes (see Stribling et al., 1992, Proc. Natl. Acad. Sci. USA 89:11277-11281, for method using non-episomal vectors). Other gene therapy approaches to kill cancer cells include expression of genes conferring drug susceptibility, such as transfection with herpes simplex thymidine kinase encoding vectors followed by ganciclovir treatment. (Culver et al., 1992, Science 256:1550-1552 used integrating vectors. Replacing the integrating vector with an episomal expression vector will enhance the level of susceptibility conferring enzyme.) Other foreign gene sequences whose expression by a patient's cells would counter-act a disease process will be apparent to those skilled in the art.

The presence of multiple copies of papovavirus-based episomes may increase expression of encoded genes compared to retroviral vectors, since only one to several copies of the retrovirus integrate per cell. Additionally, episomal DNA would be free of positional effects that may result in decreased expression from integrated vectors. The high level of transcription produced by episomal vectors of this invention may be particularly useful in antisense experiments, because high-level expression of antisense transcripts may be necessary to decrease translation of overexpressed target mRNA (Whitesell et al., 1991, Mol. Cell. Biol. 11:1360-1371).

The persistence of BKV episomes in pRP-cneoX/HT-1376 transfectants after withdrawal of selection pressure suggests that these vectors may be maintained for a reasonable period of time in human tissues. Even a transient period episomal replication may be sufficient for effective use of papovavirus episomes to treat patients with cancer. For example, wild-type anti-oncogenes capable of inducing apoptosis, such as p53 (Baker et al., 1990, Science 249:912-915; Shaw et al., 1991, Proc. Natl. Acad. Sci. USA 89:4495-4499), may need to be expressed for only a short period of time to kill transfected tumor cells. Similarly, transient expression of genes encoding susceptibility factors to chemotherapeutic agents may be effective in killing tumor cells, as has recently been demonstrated for herpes simplex thymidine kinase followed by gancyclovir treatment, and cytosine deaminase followed by 5′-fluorocytosine treatment (Culver et al., 1992, Science, 256:1550-1552; Muller et al., 1992, Proc. Natl. Acad. Sci. USA, 89:33-37). Furthermore, transient expression of cytokines, such as interleukin-4, may be effective in modulating the immune system to eliminate tumor cells (Golumbek et al., 1991, Science, 254:713-716).

The episomal vector material is generally produced by culture of recombinant or transfected cells and formulated in a pharmacologically acceptable solution or suspension, which is usually a physiologically-compatible aqueous solution, or in coated tablets, tablets, capsules, suppositories, inhalation aerosols, or ampules, as described in the art, for example in U.S. Pat. No. 4,446,128, incorporated herein by reference. Administration may be any suitable route, including oral, rectal, intranasal or by intravesicular (e.g., bladder) instillation or injection where injection may be, for example, transdermal, subcutaneous, intramuscular or intravenous.

The vector-containing composition is administered to a mammal in an amount sufficient to transfect a substantial portion of the target cells of the mammal. Determination of the amount will involve consideration of infectivity of the vector, transfection efficiency in vitro, immune response of the patient, etc. A typical initial dose for administration would be 10-1000 micrograms when administered intravenously, intramuscularly, subcutaneously, intravesicularly, or in inhalation aerosol, 100 to 1000 micrograms by mouth, or 10⁵ to 10¹⁰ plaque forming units of a recombinant vector, although this amount may be adjusted by a clinician doing the administration as commonly occurs in the administration of other pharmacological agents. A single administration may usually be sufficient to produce a therapeutic effect, but multiple administrations may be necessary to assure continued response over a substantial period of time.

Further description of suitable methods of formulation and administration according to this invention may be found in U.S. Pat. Nos. 4,592,002 and 4,920,209, incorporated herein by reference.

B. Alternative Uses for Claimed Compositions

Episomal expression vectors according to this invention also have in vitro uses. For example, episomal expression vectors may be used to enhance production of proteins that are produced in mammalian cell culture, perhaps because they must be post-translationally glycosylated (e.g., Factor VIII). Transfecting the production cell population with an episomal expression vector containing a replication cassette prepared as described above and a foreign gene encoding the desired protein will increase the expression level of the desired protein, as episomal amplification leads to high copy number of the foreign gene in the production cells.

The episomal expression vectors of this invention may be used in studies to identify potentially novel dominant oncogenes and/or anti-oncogenes that are involved in tumor progression. By constitutively expressing cDNA clones at high levels, this approach may identify new genes or genes marking biological pathways that currently have not been shown to be involved in tumorigenesis. Moreover, this assay system will permit anti-sense cDNA library screening for tumor suppressor genes, greatly simplifying the currently labor intensive methods required to identify members of this class of genes.

This may be accomplished, for instance, by transfecting nontumorigenic bladder cell lines that do not clone in soft agar with cDNA derived from tumorigenic, anchorage independent cell lines, and phenotypically screening the transfectants for the ability to grow in soft agar. The cDNAs responsible for inducing nontumorigenic cells to clone in soft agar will then be identified by shuttling the episomal vector from bladder cell transfectants to bacteria. Second generation transfection studies will subsequently confirm the ability of candidate dominant oncogenes to fully transform nontumorigenic bladder cells. In a similar fashion, cDNA libraries from nontransformed cells screened in an anti-sense orientation could theoretically identify anti-oncogenes.

It is also a discovery of the present invention that the mutant large T antigen, 107/402-T antigen, is an exceptionally efficient replication transactivator in human cells. This property of 107/402-T antigen can be employed in expression systems to produce proteins of therapeutic utility. Use of the 107/402-T antigen permits expression which continues for long periods of time and which produces large quantities of biologically active proteins.

The present invention overcomes significant limitations of the prior art. According to the present invention, human cells are genetically modified to produce very high levels of biologically functional proteins and to continue this production over long periods of time without significant cell toxicity. These human cells comprise copies of 107/402-T antigen which retain high levels of replication transactivator activity in dividing human cells. Preferably, the copies of the 107/402-T antigen are integrated. Surprisingly, 107/402-T antigen is an exceptionally efficient replication transactivator in human cells when compared with wild-type T antigen.

Also according to the present invention, either the production or activity of 107/402-T antigen in human cells can be cyclically controlled by the presence of varying concentrations of exogenous agents in the culture medium. The method of cyclically controlling replication described herein permits amplification of an episome to a level which yields high gene expression without induction of cellular toxicity. A desired protein can then be produced at high levels. Furthermore, because human cells are used in this expression system, post-translational modification of the desired protein(s) proceeds normally. Thus, the present invention provides the art with an expression system for therapeutic proteins which is useful in the pharmaceutical and biotechnology industries.

The 107/402-T antigen mutant is described in U.S. Pat. No. No. 5,624,820. Compared with the wild-type SV40 large T antigen (see Shin et al., 1975; Christian et al., 1987; Michalovitz et al., 1987; DeCaprio et al., 1988; Hanahan et al., 1989; Chen et al., 1990; Chen et al., 1992), the mutant protein contains substitutions of amino acid residues 107 (glutamic acid to lysine) and 402 (aspartic acid to glutamic acid). These amino acid substitutions prevent the 107/402-T antigen from binding to the oncogenes p53, RB, and p107, yet the mutant antigen retains the ability to activate replication of a papovavirus-based episome.

The 107/402-T antigen binds to the papovavirus origin of replication and activates the replication of adjacent DNA sequences. Under control of the 107/402-T antigen, papovavirus-based episomes replicate to thousands of copies by 2-4 days after transfection in many human cell lines. This replication is greatly enhanced compared with that observed in the presence of wild-type T antigen (Examples 4 and 5). Under control of the 107/402-T antigen, episomal copy number can range from at least 2-, 5-, 10-, 25-, 50-, 100-, 125-, 150-, 200- or 500-fold higher than episomal copy number obtained under control of a wild-type T antigen.

In one embodiment of the present invention, replication of an episome encoding the protein to be expressed is controlled by regulating transcription of the 107/402-T DNA sequence. Transcription of the DNA sequence is controlled by a minimally active promoter, which can be activated by an inducible transcriptional transregulator. The minimally active promoter prevents large amounts of 107/402-T antigen from being transcribed in the absence of an exogenous inducer of the transcriptional transregulator. Suitable minimally active promoters are, for example, the minimal CMV promoter (Boshart et al., 1985, Cell 41, 521-30) and the promoters for TK (Nordeen, 1988), IL-2, and MMTV.

An inducible transcriptional transregulator can be either a transactivator or a transrepressor. Several inducible transcriptional transactivators have been constructed, such as the hybrid tetracycline-controlled transcriptional transactivator (Gossen et al., 1992; Gossen et al. 1995), the rapamycin-controlled “gene switch” (Rivera et al., 1996), and the RU486-induced TAXI/UAS “molecular switch” (DeLort and Capecehi, 1996). Each transactivator contains a binding site for its inducer and a transcription factor domain. These inducible transcriptional transactivators bind reversibly to specific-binding regions of DNA, such as operators, and regulate an adjacent minimal promoter which is functional only when the transcription factor binds to the specific region of DNA.

Inducible repressor systems have also been developed by substituting the KRAB transcriptional repressor domain for the VP16 transactivation domain in hybrid transcription factors (Wang et al. 1997). In these systems, repression of gene transcription is linked to binding of the transcriptional repressor to the target DNA binding consensus sequence, and binding of the transcriptional repressor is controlled by suitable inducer molecules.

A transcriptional transregulator can be constructed to be either functional (“inducer-on”) or nonfunctional (“inducer-off”) in the presence of inducer. An “inducer-on” transcriptional transregulator is not functional in the absence of inducer. In the presence of inducer, the transcription factor domain of the “inducer-on” transcriptional transregulator binds to the specific-binding DNA region and activates the minimally active promoter. An “inducer-off” transcriptional transregulator functions in the absence of inducer. In the presence of inducer, the transcription factor domain of the “inducer-off” transcriptional transregulator does not bind to the specific-binding DNA region and does not activate the minimally active promoter. DNA sequences encoding either type of inducible transcriptional transregulator can be used to practice this invention.

DNA sequences encoding the 107/402-T antigen, a minimally active promoter, and an inducible transcriptional transregulator can be located on the same DNA construct or can be encoded by separate DNA constructs. A DNA construct can also encode any two of the three elements. Optionally, the DNA sequences encoding the transcriptional transregulator and the DNA sequence encoding the 107/402-T antigen can be on an episome. The episome can comprise a papovavirus origin of replication and a restriction enzyme site for insertion of a coding sequence of a desired protein. Alternatively, the papovavirus origin of replication and restriction enzyme site can be on an episome separate from the DNA constructs encoding the 107/402 antigen, the minimally active promoter, and the inducible transcriptional transregulator. The episome can also comprise a promoter which regulates transcription of the coding sequence of the desired protein. Individual DNA constructs or episomes can be introduced into a cell together or separately, as is desired.

Expression vectors can be constructed containing one or more copies of a particular DNA construct. Many suitable vectors are available from commercial suppliers, such as Stratagene, GIBCO-BRL, Amersham, and Promega, as well as from noncommercial sources such as the American Type Culture Collection (ATCC), 10801 University Blvd., Manassas, Va. 20110-2209. Suitable vectors may also be constructed in the laboratory using standard recombinant DNA techniques (Sambrook et al., 1989; Glover, 1985; Perbal, 1984). The sequences can be synthesized chemically or can produced by recombinant DNA methods.

Methods of transfecting DNA into human cells are well known in the art. These methods include, but are not limited to, transferrin-polycation-mediated DNA transfer, transfer with naked or encapsulated nucleic acids, liposome-mediated cell fusion, intracellular uptake of DNA-coated latex beads, protoplast fusion, viral infection, electroporation, and calcium phosphate-mediated transfection. Integration of the DNA sequences encoding the inducible transcription transregulator and the 107/402-T antigen into the host cell's DNA can be facilitated by providing nucleotides at the 3′ or 5′ ends of these DNA sequences which are homologous to and therefore recombine with the host cell DNA. One or more copies of each DNA sequence or episome can be integrated into the genome of the host cell, as desired.

The host cell can be any human cell. Preferably, the host cell is capable of dividing and being maintained in vitro, such as HT-1376 (bladder carcinoma), HepG2 (hepatoma), HEK 293 (human embryonic kidney), HT1080 (fibrosarcoma), HeLa (cervical carcinoma), Hs68 (fibroblasts), RAJI (lymphoma), SW480 (colon cancer), 5637 (bladder carcinoma), MCF-7 (breast carcinoma), or HuNS1 (myeloma) cells. Preferred host cells are those which are particularly well-suited for protein secretion, such as myeloma cell lines. Many of these cell lines, together with instructions on how to culture them, are available from the ATCC. Suitable methods for maintaining cell lines in culture are also well known in the art (see Freshney, 1986).

In addition to containing the minimally active promoter, the DNA sequences encoding the 107/402-T antigen, and an inducible transcriptional transregulator, the host cell can contain an episome. The episome comprises a papovavirus origin of replication, a DNA sequence encoding the desired protein to be expressed, a promoter which is functional in the host cell, and a multiple cloning site for insertion of the protein coding sequence, or transgene (see, for example, Walter and Blobel, 1982; Caras and Weddell, 1989, Science 243, 1196-98). According to the invention, transgene expression can be increased at least 2-, 3-, 4-, or 5- fold or more over expression levels achieved using an expression vector encoding wild-type T antigen.

The protein encoded by the transgene or protein coding sequence can be, for example, any protein of therapeutic utility, including but not limited to a structural protein, an anti-angiogenic or pro-angiogenic factor, a transcription factor, a cytokine, a neuropeptide, a ligand for a cell surface receptor, an enzyme, a growth factor, a receptor for a ligand, an antibody, a hormone, a transport protein, a storage protein, a contractile protein, or a novel engineered protein. The protein can be one which is normally encoded by an endogenous gene in the host cell or can be a protein not normally found in the host cell. The protein can be identical to a naturally occurring protein or can contain modifications to alter its physicochemical properties, such as stability, activity, affinity for a particular ligand or receptor, antigenicity, therapeutic utility, or ability to be secreted from the host cell. The protein can also be a fusion protein comprising two or more protein fragments fused together by means of a peptide bond. The fusion protein can include signal peptide sequences to cause secretion of the protein into the culture medium. Such sequences are well known in the art.

The promoter can be any promoter which is functional in the selected host cell. Highly active promoters, such as the regulatory region of elongation factor-1α (Guo et al., 1996), are preferred. Multiple cloning sites are well known in the art and can be inserted into the episome using standard recombinant DNA techniques.

The episome also comprises a papovavirus origin of replication to which the 107/402-T antigen binds. In a preferred embodiment, the origin of replication is an SV40 or a BK origin of replication. The sequence of the SV40 origin of replication is taught in Subramanian et al., 1977; Reddy et al. 1978; Fiers et al., 1978; and Van Heuverswyn et al., 1978. The sequence of the BK origin of replication is disclosed in Yang et al. (1979) and Deyerle et al. (1989).

Those of skill in the art can select suitable episomes for use in this protein expression system from those available commercially or noncommercially, such as from the ATCC. Alternatively, one can synthesize an episome in the laboratory using standard recombinant DNA techniques. Episomes can also contain a selectable marker, such as the neomycin phosphotransferase gene or antibiotic resistance genes.

In one embodiment of the invention, the host cell is cultured in a medium which is suitable to maintain the particular cell type being used. The cell is contacted with an inducer of the inducible transcriptional transregulator. The inducer can be a component of the cell culture medium or can be added separately. In a preferred embodiment, the inducible transcriptional transregulator is a hybrid tetracycline-controlled transcriptional transactivator. Tetracycline or a tetracycline derivative such as oxytetracycline, chlortetracycline, anhydrotetracycline, or doxycycline, is added to the culture medium to cause the transactivator to regulate transcription of the DNA sequence encoding the 107/402-T antigen.

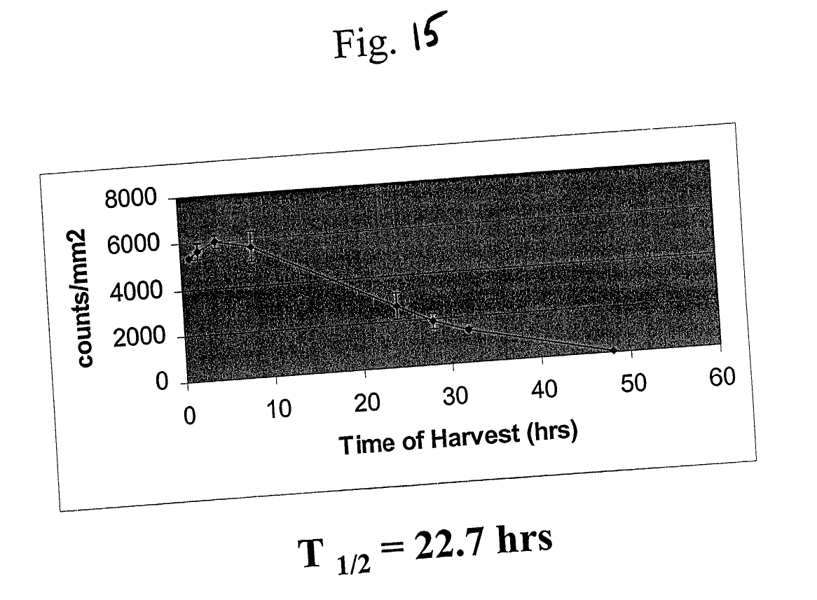

The concentration of inducer is selected by routine experimentation to result in an episome copy number for the particular cell line which results in maximal expression of the protein without cellular toxicity. Appropriate copy numbers range from at least 10 to at least 100, at least 100 to at least 1,000, at least 1,000 to at least 10,000, at least 10,000 to at least 50,000, at least 50,000 to at least 100,000, or at least 100,000 to at least 500,000 copies or more of the plasmid per cell. Plasmid copy number can be measured, for example, by Southern blot (Cooper and Miron, 1993). For tetracycline or its derivatives, effective concentrations range from at least 1 pg/ml to at least 1 μg/ml. For rapamycin, suitable concentrations range from at least 500 pM to at least 2 nM to at least 10 nM to at least 100 nM. The half-maximal concentration for inhibition using doxycycline, for example is approximately 0.01 ng/ml (FIG. 15). Concentrations of RU486 which can be used effectively range from at least 1 nM to at least 100 nM.

Inducer concentration can be varied over time to achieve suitable copy numbers per cell. For example, inducer can be present continuously for 1-3 days or for 1-6 days and then removed entirely, for example by changing the medium. Alternatively, medium can be changed every 2-3 days and the concentration of inducer can be varied, for example, by one-half or one-tenth. The precise variation regimen will depend on the cell being used and the stability of the inducer under particular culture conditions. These parameters can be determined by routine experimentation. Thus, one skilled in the art can empirically vary the inducer regimen to maximize the output of transgene expression for any given construct of interest. The optimal regimen will be based, in part, on potential toxicities of the desired protein to the producer cell line, the extent to which transcription factors are in limited concentration as they bind to amplified promoter regions in episomes encoding the desired protein, and other factors which may limit the inherent production capabilities of the producer cell line.

The invention also provides a kit for expressing a desired protein by regulating transcription of the 107/402-T antigen. The kit comprises a human cell and a first episome. The human cell can be any of the cells described above. The first episome comprises a papovavirus origin of replication, such as the SV40 or BK origins of replication, to which the 107/402-T antigen binds. The first episome is used as a vector for a coding sequence for the desired protein. The coding sequence for the desired protein can be inserted into the first episome using standard recombinant DNA techniques. The first episome can also contain an active promoter, for example the regulatory region from elongation factor-1α. A restriction enzyme site or multiple cloning site can be included in the first episome to permit incorporation of the protein coding sequence, or the first episome can be provided with a coding sequence for a desired protein already inserted.

The human cell also contains one or more copies of a first DNA sequence encoding an inducible transcriptional transregulator, a minimally active promoter, and a second DNA sequence encoding the 107/402-T antigen. The DNA sequences encoding the inducer transcriptional transregulator and the 107/402-T antigen can be integrated into the genome of the cells or can be on the first episome or a second episome.

In another embodiment of the invention, replication of the episome encoding the protein to be expressed is controlled by regulating the activity of the 107/402-T antigen, by means of a “protein switch.” This regulation is accomplished by providing the cell with a fusion protein comprising two protein segments fused together by means of a peptide bond. The first protein segment comprises the 107/402-T antigen. The second protein segment comprises a mutant progesterone receptor. The mutant receptor includes a hormone binding domain that binds only synthetic antiprogestins, such as RU486. Other segments of the human progesterone receptor have comparable properties (DeLort and Capecchi, 1996). Mutant progesterone receptors include progesterone receptors which comprise amino acids not normally present in a progesterone receptor, truncated progesterones, and the like. One sequence of a mutant receptor is taught in Vegeto et al. (1992). This particular mutant progesterone receptor lacks 54 authentic C-terminal amino acids and includes 12 novel amino acids at the C-terminal.

In the absence of antiprogestin, the mutant progesterone receptor in the fusion protein interferes with the ability of the 107/402-T antigen to function as a replication transactivator. In the presence of RU486, however, the conformation of the mutant progesterone receptor changes and 107/402-T antigen becomes functional. Replication of an episome which contains a papovavirus origin of replication can then take place. Thus, the fusion protein functions as a protein switch which regulates the replication activating activity of 107/402-T antigen. Within the fusion protein, the hormone binding domain of mutant progesterone receptor can be located at either the C-terminal or the N-terminal of the 107/402-T antigen, or in the middle of the 107/402-T antigen molecule.

A vector for expressing the fusion protein can be constructed using recombinant DNA techniques available in the art. The vector preferably comprises an active promoter for expressing large quantities of the fusion protein. A promoter such as the CMV immediate early promoter-enhancer, or a highly active human promoter such as the regulatory region from elongation factor-la, can be used for this purpose. Alternatively, promoters which are specifically active in tumor cells, for example oncofetal promoters such as the α-fetoprotein promoter (Huber et al., 1991) or CEA promoter (Osaki et al., 1994), can be used to regulate expression of the fusion protein. The vector can be introduced into a human cell and stably integrated into the host DNA using the methods described above. Suitable host cells for use in this embodiment are those described above. The host cell can contain or can later be a recipient of an episome containing a papovavirus origin of replication and a DNA sequence encoding a desired protein, as described above. The promoter which regulates transcription of the DNA sequence encoding the fusion protein can also regulate transcription of the DNA sequence encoding the desired protein, for example, by including between the two coding sequences an internal ribosome entry site, as is known in the art. Alternatively, the episome can contain a separate promoter for regulating transcription of the DNA sequence encoding the desired protein.

For in vitro protein production, the host cell is grown in an appropriate culture medium. In a preferred embodiment, RU486 is added to the cell. Other antiprogestins, such as Onapristone, Org31710, or ZK112993, can also be used. The antiprogestin can be a component of the culture medium or can be added separately. The concentration of antiprogestin is selected by routine experimentation to result in an episome copy number for the particular cell line which results in maximal expression of the protein without cellular toxicity. Appropriate copy numbers, as measured, for example, by Southern blot (Cooper and Miron, 1993), range from at least 10 to at least 100, at least 100 to at least 1,000, at least 1,000 to at least 10,000, at least 10,000 to at least 50,000, at least 50,000 to at least 100,000, or at least 100,000 to at least 500,000 or more copies of the plasmid per cell. The concentration of antiprogestin which results in appropriate plasmid copy numbers for a particular cell type ranges from at least 1 nM to at least 10, 25, 50, 75, or 100 nM. The concentration of antiprogestin can be varied over time to achieve suitable copy numbers per cell.

The invention also provides a kit for expressing a desired protein by regulating activity of the 107/402-T antigen. The kit comprises a human cell and an episome. The human cell can be any of the cells described above and contains a one or more copies of a DNA sequence encoding a 107/402-T-mutant progesterone receptor fusion protein. Optionally, the DNA sequence encoding the fusion protein can be integrated into the cell's genome. Expression of the fusion protein is controlled by an active promoter, as described above. The episome comprises a papovavirus origin of replication to which the 107/402-T antigen binds, such as an SV40 or BK origin of replication. The episome is used for insertion of a coding sequence for the desired protein and can also be integrated into the genome of the cell if desired. The coding sequence for the desired protein can be inserted into the episome using standard recombinant DNA techniques. One or more restriction enzyme sites or a multiple cloning site can be included in the episome to permit incorporation of the protein coding sequence. Optionally, the human cell of the kit can comprise the episome. Transcription of the coding sequence of the desired protein can be regulated by the promoter which regulates expression of the fusion protein or by a separate promoter, as described above.

### EXAMPLE 1

BKV Episomal Plasmids can Stably Replicate in HT-1376 Cells

Plasmids

pRP-cCATX and pRP-cncoX are BKV episomal plasmids that contain a 3.2-kb fragment of BKV encoding the origin of DNA replication and the large T antigen (Grossi et al., 1988). pRP-cCATX encodes the chloramphenicol acetyltransferase (CAT) gene driven by the SV40 early promoter, whereas pRP-cncox encodes the neomycin resistance gene [phosphotransferase APH(3′)II from transpposon Th5] driven by the SV40 early promoter. pSV2CAT/220.2 is a derivative of pSV2CAT containing the EBV episomal element (Haver et al., 1989). pSV2NEO encodes the neomycin resistance gene driven by the SV40 early promoter (Southern and Berg, 1982). pSV2CAT encodes the CAT gene transcriptionally regulated by the SV40 early promoter, and pSVOCAT is a derivative of pSV2CAT lacking the SV40 early promoter (Gorman et al., 1982).

To evaluate if a BKV episomal plasmid can stably replicate in bladder carcinoma cells, HT-1376 cells were transfected with pRP-cneoX, a derivative of pSV2NEO containing a 3.2 kb episomal element consisting of the BKV origin of DNA replication and the BKV large T antigen (Grossi et al., 1988, Arch. Virol., 102:275-283).

Transfection and Selection

A total of 1.5×10⁶ cells in 60-mm dishes were transfected using 10 μg of plasmid DNA and 40 μg of lipofectin in 3 ml of Optimem (Gibco-Bethesda Research Labs, Gaithersburg, Md.). Following 6 hours of incubation, DMEM was added with supplemental fetal calf serum to obtain a final concentration of 10%. Two days after transfection, cells were trypsinized and seeded in six-well plates, and 24 hours later 200 μg/ml G418 was added to the media to initiate selection.

Southern Blots

DNA from transfected cells was evaluated by Southern blot after 71 days of G418 selection, and the Southern blots were probed with ³²P-labelled pRP-cneoX. Low-molecular-weight DNA (Hirt supernatant DNA) was prepared from HT-1376 transfectants as described by Hirt, 1967, J. Mol. Biol. 26:365-369). Total cellular DNA was removed from CsCl gradients and purified as previously described (Davis et al., 1986, “Basic Methods In Molecular Biology”, Elsevier Science Publishing, NY, pp. 130-135). Hirt supernatant and digested total cellular DNAs were electrophoresed in 0.7% agarose gels, transferred to Nytran membranes (Schleicher & Scheull, Keene, NH), hybridized to ³²P-labeled random primed probes, and washed to a final stringency of 0.2×saline sodium citrate (SSC)/1.0% sodium dodecyl sulfate (SDS) at 65° C.

A. Episomal Replication in Stable Transfectants

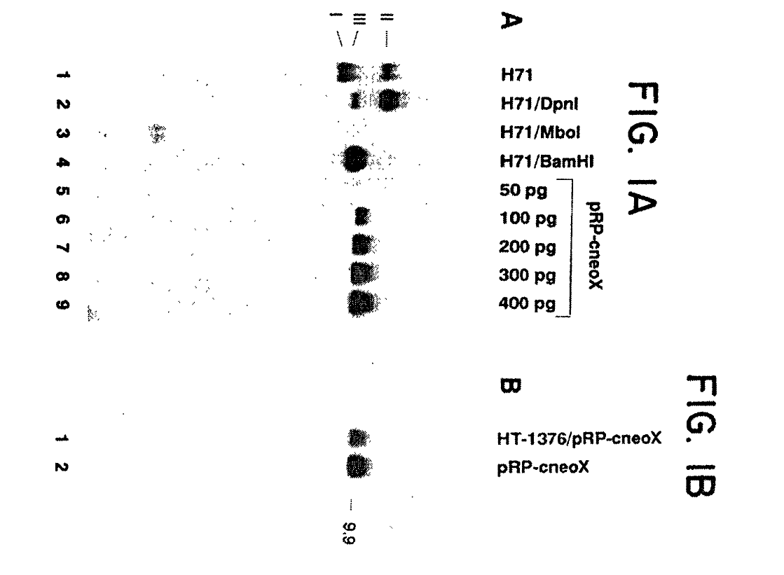

Low molecular weight DNA (Hirt supernatant DNA) derived from these stable transfectants was subjected to Southern blot analysis, and the data, presented below in FIG. 1, Panel A, shows episomal plasmids. Plasmid forms II (nicked circular) and III (supercoiled) are evident in lane 1, indicating that free plasmid DNA is present in these transfectants. The pRP-cneoX episome in these Hirt supernatants is the same size as plasmid controls, indicating no detectable rearrangements or internal deletions of the episome as it is passaged in HT-1376 cells.

To confirm that this plasmid DNA is newly replicated episomal DNA, lanes 2 and 3 show Hirt supernatant DNA was digested with DpnI and MboI, respectively (Pipas et al., 1983, Mol. Cell. Biol. 3:203-213). DpnI will cleave the GATC recognition site when both adenine bases are methylated, a feature of plasmid DNA synthesized in DNA adenine methylase (DAM) positive bacteria (input DNA). In contrast, MboI will cleave the GATC recognition sequence when adenine bases are not methylated. Since human cells lack the DAM enzyme, MboI digestion of Hirt supernatant DNA indicates that the episome replicated extrachromosomally. The lack of restriction fragments following DpnI digestion and the complete cleavage of Hirt supernatant DNA following MboI digestion confirms that the plasmid DNA present in these transfectants is newly replicated, episomal DNA.

B. There is a High Copy Number of BKV Episomes in HT-1376 Transfectants

To determine the copy number of episomes per cell, BamHI-digested Hirt supernatant DNA and a standard curve consisting of increasing amounts (50-400 pg) of BamHI-digested, linearized pRP-cneoX plasmid was evaluated by Southern blot analysis (FIG. 1, Panel A, lanes 4-9). Densitometric analysis of these bands indicates that there is approximately 500 pg of pRP-cneoX per 3×10⁵ cells, or approximately 150 copies of the episome per cell. This copy number is higher than reported for most other episomal vectors. For example, a typical copy number of Epstein-Barr virus-based episomal vectors in lymphoid cells is approximately 10 to 50 per cell (Yates et al., 1985). The high copy number of BKV episomes in HT-1376 cells suggests that the steady state level for transcription of genes encoded by such a vector is likely to be very high, and that this vector will be efficiently transferred to the progeny of these bladder transfectants during cellular division. Both of these possibilities were evaluated in the experiments which follow.

C. There is no evidence that pRP-cneoX integrates into HT-1376 genomic DNA.

To evaluate if pRP-cneoX also integrates into HT-1376 DNA, total cellular DNA from HT-1376 transfectants was digested with BamHI and evaluated by Southern blot analysis (FIG. 1, Panel B, lane 1). A single band of 9.9 kb, identical in size to BamHI-digested pRP-cneoX plasmid control (lane 2), is consistent with linearized episomal plasmid. Although a 9.9 kb band might also be due to tandem copies of integrated pRP-cneoX plasmid, the absence of other restriction fragments in this analysis indicates that the frequency of integration of the pRP-cneoX episome is very low, beneath the limit of resolution of this assay. This finding is important, since a low frequency of integration of BKV episomes will limit chance insertional activation of proto-oncogenes or insertional inactivation of tumor suppressor genes.

### EXAMPLE 2

The Steady State Level of Transcription of the Neomycin Resistance Gene in pRP-cneoX Transfectants is 20-Fold Higher than in pSV2NEO Transfectants

In order to evaluate the potential advantages of utilizing a BKV episomal vector compared to standard plasmid vectors, HT-1376 cells were transacted with pSV2NEO, a plasmid that will be unable to replicate extrachromosomally in HT-1376 cells in the absence of exogenous large T antigen. Following transfection, cells were selected in neomycin, and 5 to 10 clones were combined in each of 3 different pools (a, b, c). Southern analysis of genomic DNA derived from these pools indicated that there were approximately 5 copies of pSV2NEO per cell (data not shown).

To evaluate whether BKV episomal expression vectors can produce high levels of transcription, the steady state level of neomycin resistance gene mRNA from pRP-cneoX and pSV2NEO HT-1376 transfectants were compared by Northern blot analysis. In both plasmids, transcription of the neomycin resistance gene is regulated by the SV40 early promoter.

Northern Blots

Total cellular RNA was prepared by the CsCl isothiocyanate method of Chirgwin et al. (1979, Biochem, 18:5294-5299). Twenty micrograms of total cellular RNA were electrophoresed in 1% agarose formaldehyde gels, transferred to Nytran membranes, hybridized to ³²P-labeled random primed probes, and washed to a final stringency of 0.1×SSC/1.0% SDS at 65° C., as previously described (Cooper et al., 1990, Cell Growth Differen. 1:149-159).

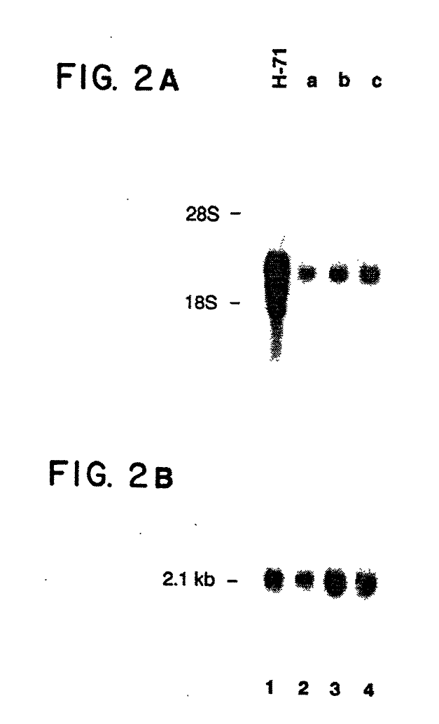

In FIG. 2, Panel A, 20 μg of total cellular RNA from pRP-cneoX (lane 1) and pSV2NEO (pool a, lane 2; pool b, lane 3; pool c, lane 4) transfectants were probed with a radiolabeled BamHI/HindIII fragment of pSV2NEO encoding the neomycin resistance gene. To confirm that approximately equal amounts of mRNA were loaded in each lane, this blot was reprobed with a radiolabeled beta-actin probe (FIG. 2, Panel B).

Densitometric analysis of these data indicates that there are approximately 20-fold higher levels of steady state expression of the neomycin resistance gene in the pRP-cneoX episomal transfectants compared to the pSV2NEO transfectants. This difference is presumably due in part to the higher copy number of pRP-cneoX (150 copies) compared to pSV2NEO (5 copies) in HT-1376 transfectants. These data indicate that BKV episomal expression vectors can achieve substantially higher levels of transcription of a transfected gene than plasmid or retroviral vectors that depend on integration for stable expression.

### EXAMPLE 3

BKV Episomal Vectors are Efficiently Transferred to the Progeny of Bladder Transfectants during Cellular Division

In order to effectively use an episomal expression vector for cDNA library screening, the episome must be efficiently transferred from one transfected cell to its progeny during cell division. A high copy number of episomes per cell may be predictive of efficient vertical transfer since, in this circumstance, it would be unlikely that all episomes would partition to a single daughter cell. Since the parent cell line, HT-1376, clones in soft agar, it was possible to directly evaluate the vertical transfer efficiency of pRP-cneoX in HT-1376 transfectants by plating these cells in soft agar in the presence or absence of neomycin. As a positive control for efficient vertical transfer, pSV2NEO HT-1376 transfectants (pool b), in which the neomycin resistance gene is integrated into HT-1376 DNA, was plated in soft agar in the presence or absence of neomycin.

These results are presented below in Table 1. The parent cell line, HT-1376, clones in soft agar in the absence of neomycin with an efficiency of 0.83%, and does not clone in 200 pg/ml of neomycin, a concentration previously shown to kill these cells after 14 days of incubation. The ratio of soft agar cloning efficiencies with and without neomycin for both types of transfectants is essentially identical, demonstrating efficient transfer of the episome during cell division. In addition, there was no difference in the size of soft agar colonies of HT-1376 pRP-cneoX transfectants grown in the absence or presence of neomycin (data not shown), further evidence in support of efficient vertical transfer of BKV episomes in these cells.

TABLE 1
Vertical transfer efficiency of pRP-cneoX in HT-1376 bladder cells.

SOFT AGAR CLONING EFFICIENCY*

| | no neomycin | 20 μg/ml neomycin |
|---|---|---|
| ht-1376 | 0.83 +/− 0.09 | 0 |
| ht-1376/pSV2NEO | 0.75 +/− .0.01 | 0.65 +/ 0.16 |
| ht-1376/pRP-cneoX | 0.82 +/− 0.07 | 0.59 +/− 0.17 |

*Cloning efficiency is expressed as the number of soft agar colonies divided by the number of cells plated, tabulated as a percentage. 10⁵ cells were plated per dish and colonies were scored after 3 weeks of growth. Values are the mean of triplicate determinations +/−standard deviation.

### EXAMPLE 4

BKV Episomes Persist in Bladder Transfectants after Withdrawal of Selection Pressure

The high copy number of pRP-cneoX in HT-1376 transfectants and the efficient vertical transfer of this episome to the progeny of these transfectants raised the possibility that pRP-cneoX may be maintained in these cells for several weeks or months without selection pressure. To evaluate the persistence of pRP-cneoX in HT-1376 cells in the absence of selection pressure, these transfectants were grown in complete media without G418, and at various times Hirt supernatant DNA was prepared for Southern analysis. In FIG. 3, lanes 3-6, Hirt supernatant DNA is analyzed from transfectants grown in the absence of G418 for 16, 34, 47, and 64 days, respectively. The sample in lane 2, from cells cultured in the presence of G418 after 122 days of selection, coincides with the 34 day time point during the course of G418 withdrawal, serving as a reference for comparison.

In panel A, this blot was probed with radiolabeled pRP-cneoX. The episome copy number is maintained at essentially unreduced levels following 16 days of withdrawal of G418, and then appears to transiently fall to approximately 10% of the control level by 34 days of selection. Unexpectedly, the episome copy number then increases back to control levels by 64 days of G418 withdrawal.

To evaluate if differences in episomal copy number are due to random inefficiencies in preparation of Hirt supernatant DNA, this blot was rehybridized to a probe for mitochondrial DNA (panel B). The essentially equal amount of mitochondrial DNA in each Hirt supernatant demonstrates that extracts from comparable numbers of cells were loaded in each lane, and that the copy number of pRP-cneoX is indeed maintained at high levels after 64 days of growth in the absence of selection pressure. These data indicate that brief periods of growth of bladder cell transfectants in the absence of G418 will be unlikely to result in loss of episomal plasmid DNA.

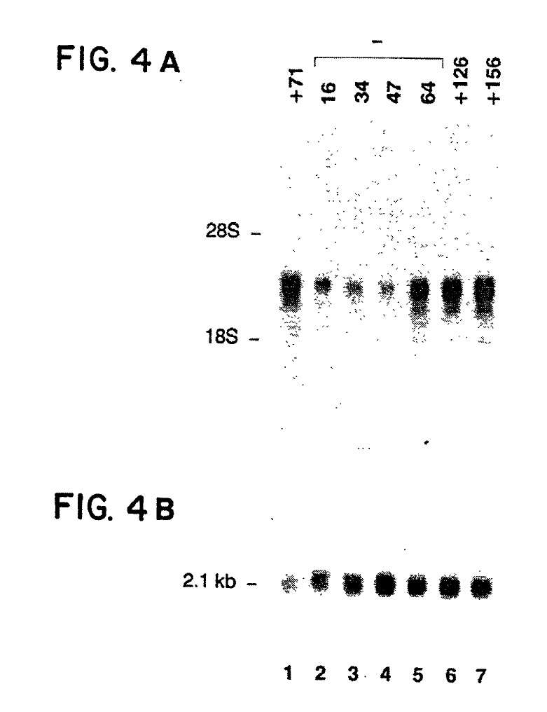

The finding that BKV-derived episomes are maintained at high copy numbers after 2 months of growth in the absence of G418 was unanticipated, since EBV and SV40-derived episomes are usually lost from stable transfectants after 2-4 weeks of growth in the absence of selection pressures (Yates et al., 1984; Hamber et al. 1988; Chittenden et al., 1991). To confirm further that pRP-cneoX is maintained in HT-1376 cells in the absence of G418, we evaluated expression of the neomycin resistance gene during this time course (FIG. 4A). Comparable to the episomal copy number (FIG. 3A), we observed a transient fall in neomycin resistance gene expression followed by return to essentially control levels by 64 days of G418 withdrawal. Equivalent loading of RNA in each lane is demonstrated by rehybridizing this blot with a probe for β-actin (FIG. 4B). These data strongly argue that BKV-derived episomes can be maintained at a high copy number in bladder carcinoma cells in the absence of selection pressure.

### EXAMPLE 5

BKV Episomal Vectors can be Shuttled between HT-1376 Bladder Cell Transfectants and Bacteria

An important advantage of episomal expression vectors compared to standard plasmid or viral constructs is the ability to shuttle the episome from stable transfectants into competent bacteria. Hirt supernatant DNA from HT-1376/pRP-cneoX stable transfectants was used to electroporate DH10B E. coli. Of 12 minipreps analyzed, 10 had unrearranged episomal plasmid (data not shown), consistent with the findings presented in FIG. 1. For the two colonies having minor rearrangements, it is unclear whether these changes occurred in the bladder cell transfectants or during passage in bacteria.

### EXAMPLE 6

Development of a Replication-Competent, Transformation-Negative Hybrid SV40/BKV-Derived Episomal Expression System

The BKV episomal vectors used in Examples 1-5 contained a 3.2 kb fragment of BKV including the origin of DNA replication and the BKV large T antigen (BK-T) transcriptionally regulated by the BKV early promoter. It is expected that BK-T would induce soft agar growth based on its ability to complex wild-type p53 and RB (Mann et al., 1984; Dyson et al., 1990). As described below, this vector system was modified by substituting replication-competent, transformation-negative SV40 large T antigen mutants for BK-T. This strategy has been successful, and clones of bladder cell transfectants expressing high levels of SV-T mutant protein remain nontumorigenic and induce plasmids containing SV40 or BKV DNA origins to replicate extrachromosomally.

Wild-type SV-T and each SV-T mutant have been subcloned into the pRc/CMV expression vector (Invitrogen). In this vector, expression of SV-T is regulated by the efficient CMV promoter-enhancer which is active in all bladder cell lines tested. Additionally, lack of down-regulation by SV-T protein makes the CMV promoter-enhancer a good choice for these studies. Wild-type and mutant SV40 large T antigen cDNA were subcloned into the multiple cloning site of the CMV promoter-enhancer transcriptional cassette in the pRC/CMV expression vector (Invitrogen) using a two-part strategy. First, pRC/CMV.T and pRC/CMV. 107-T were constructed. cDNAs encoding wild-type SV40 large T antigen and the 107-T (K1) mutant (Kalderon et al.) were initially available as subcloned fragments in the unique BamHI site of the pSG5 vector. These vectors were digested with BamHI, fragments containing T antigen cDNA were gel purified, and 3′ termini were filled in using the Klenow fragment of DNA polymerase 1. Phosphorylated XbaI linkers were added, followed by XbaI digestion and gel purification. T antigen cDNA clones were then ligated into XbaI-digested, calf intestine alkaline phosphatase-treated pRc/CMV. Orientation of the T antigen cDNA clones was determined by digestion with XmnI and PstI.

Secondly, pRC/CMV.T and pRC/CMV.107-T were modified to produce pRC/CMV.402-T and pRC/CMV. 107/402-T. A clone of SV40 encoding the codon 402 aspartic acid to glutamic acid mutation in SV40 large T antigen was obtained from the laboratory of Dr. D. Simmons. This clone was digested with HpaI, and a 1067 base pair C-terminal fragment of T antigen was gel purified. Similarly, pRC/CMV.T and pRC/CMV-107-T were digested with HpaI, and the large 6.4 kb fragments were gel purified. The C-terminal HpaI fragment from 402-T was then ligated with calf intestine alkaline phosphatase-treated parent vectors to produce pRC/CMV.402-T and pRC/CMV.107/402-T. Orientation of the T antigen cDNA clones was determined by digestion with A1wNI.

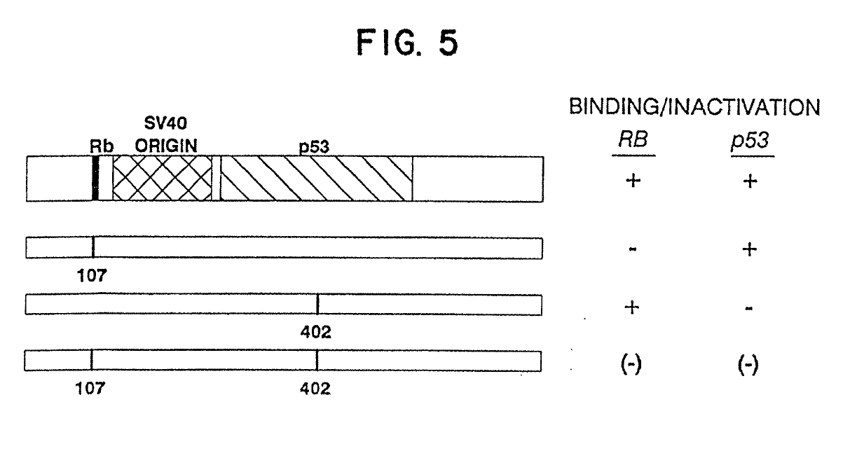

Partial DNA sequence analysis of these constructs confirms that these SV-T mutants do indeed contain the predicted point mutations. FIG. 5 shows the location of point mutations in replication competent, transformation negative SV40 large T antigen (SV-T) mutants. The p53 and RB binding characteristics of 107/402-T, indicated in parenthesis, are predicted results.

A. Expression of large T antigen mutants by nontumorigenic 5637 cell line.

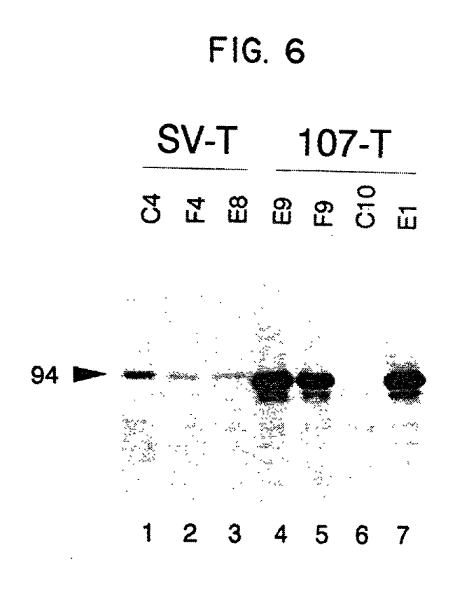

Because the 5637 cell line is nontumorigenic and has mutations in both p53 and RB, it seemed likely that expression of either wild-type or mutant SV-T protein would not induce tumorigenic properties. 5637 was therefore chosen for initial transfection studies. 5637 was transfected with pRc/CMV.SV-T and pRc/CMV.107-T using the lipofection method (Felgner et al., 1987, Proc. Natl. Acad. Sci. USA 84:7413-7417), stable transfectants were selected in G418, and single cell clones were isolated using cloning cylinders. Shown in FIG. 6 below is a Western blot analysis of representative single cell clones for SV-T and 107-T expression. A protein of 94 kD is detected in these studies, identical in size to T antigen produced in COS-7 control cells. All three SV-T clones have moderate levels of T antigen expression (lanes 1-3). Three out of 4 107-T clones had high levels of T antigen expression (lanes 4, 5, and 7), with clone C10 having no detectable T antigen expression (lane 6). Expression of mutant large T antigen was similarly observed for 5637 cells transfected with pRc/CMV.402-T and pRc/CMV.107/402-T.

B. Expression of mutant large T antigen does not induce tumorigenic properties in susceptible cells.

Clones of SV-T and 107-T 5637 transfectants have been evaluated for the ability to grow in soft agar, to form foci in tissue culture, and to form tumors in the flanks of nude mice. Three 5637 clones were chosen for these initial studies (see FIG. 6): C4 (SV-T expressor); C10 (107-T non-expressor); and E1 (107-T expressor).

Soft Agar Cloning

Transformed cells were trypsinized and then passed through a 30-micron nylon filter (Tetko, Lancaster, N.Y.) to achieve a single cell suspension. The bottom layer of agar consisted of low glucose DMEM supplemented with 10% fetal calf serum and 0.6% Seaplaque agarose (FMC BioProducts, Rockland, Me.). The top layer contained serial dilutions of cells ranging between 10³ and 10⁶ in low-glucose DMEM supplemented with 10% fetal calf serum and 0.3% Seaplaque agarose. Aggregates of cells greater than 125 μm in diameter (˜50 cells) were scored as colonies, and dishes were observed for at least 1 month after plating. These data are summarize below in Table 2.

The parent nontumorigenic 5637 cell line does not clone in soft agar, form foci in tissue culture, and only 1/12 nude mice inoculated formed a tumor after a prolonged latency period. The moderate SV-T expressor, C4, remains nontumorigenic in nude mice and does not clone in soft agar. Interestingly, this clone forms microscopic foci, although does not form macroscopic foci. As expected, clone C10, the 107-T non-expressor, is nontumorigenic with an identical profile as the parental 5637 cell line. The high level 107-T expressor, E1, has an identical profile as C4, with formation of microscopic foci, but no growth in soft agar or in nude mice. These data demonstrate that we have successfully expressed T antigen protein in nontumorigenic bladder carcinoma cells without inducing anchorage independence or tumor formation in nude mice.

TABLE 2
5637 bladder carcinoma cells transfected with wild-type (SV-T) or mutant
(107-T) SV40 large T antigens remain nontumorigenic.

| | T ANTIGEN | FOCUS | SA (%) | TUMORS | LATENCY |
|---|---|---|---|---|---|
| Parental | − | − | 0 | 1/12 | 12 |
| SV-T C4 | + | +/− | 0 | 0/6 | — |
| 107-T C10 | − | − | 0 | 0/6 | — |
| 107-T E1 | ++ | +/− | 0 | 0/6 | — |

T ANTIGEN, level of T antigen expression (−none, +moderate, ++high); FOCUS, focus formation (−none, +/−microscopic foci); SA, soft agar growth; TUMORS, # tumors/total number of nude mice inoculated; LATENCY, time (weeks) to achieve tumor volume >250 mm³

Expression of wild-type SV40 T antigen will confer soft agar growth in a suitable recipient cell line. Since T24 cell line has wild-type RB (and mutant p53), expression of SV-T or 402-T is expected to inactivate RB protein. We have characterized tumorigenic properties in clones of T24 cells expressing moderate levels of SV-T (A7) and 402-T (G1). Whereas the parent T24 cell line does not clone in soft agar, A7 and G1 have cloning efficiencies of 0.10%±0.02% (se) and 0.12%±0.04%, respectively. These findings indicate that the T antigen proteins expressed in these transfectants are biologically functional molecules retaining transformation properties. In contrast, a single cell clone of T24 expressing high levels of 107/402-T does not grow in soft agar.

C. 107-T can drive replication of the SV40 DNA origin.

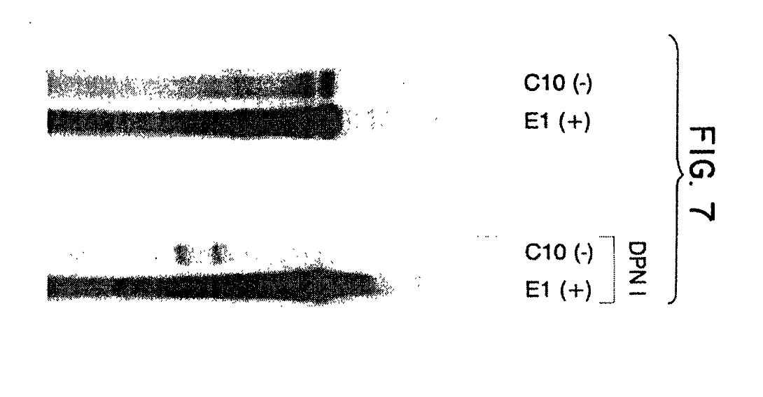

A biological function expected for SV-T and 107-T is the ability to drive replication of the SV40 DNA origin. To evaluate replication activity of 107-T, 5637 clones C10 and E1 were transfected with pSV2CAT, a plasmid containing the SV40 DNA origin. Four days after transfection, Hirt supernatant DNA was prepared and evaluated for evidence of episomal replication, as illustrated in FIG. 7.

Extrachromosomal replication can be assayed by determining if Hirt supernatant DNA is partially resistant to digestion by DpnI. Whereas plasmid DNA prepared in DNA adenine methylase positive bacteria are methylated at adenine nucleotides at the sequence GATC, mammalian cells lack this enzyme, and hence human DNA is resistant to digestion by DpnI. As observed in FIG. 7, Hirt DNA prepared from the C10 5637 clone (107-T non-expressor) fails to support episomal replication, since pSV2CAT is efficiently digested by DpnI. In contrast, Hirt DNA prepared from the E1 5637 clone (107-T expressor) is largely resistant to digestion by DpnI, indicating that pSV2CAT is replicating extrachromosomally in these transfectants. These data demonstrate that SV40 large T antigens expressed in bladder cell transfectants are biologically functional molecules, possessing replication activity.

### EXAMPLE 7

Construction of the 107/402-T Antigen Mutant

Wild-type SV40 large T antigen cDNA was isolated from plasmid pSG5-T as a 2.1 kb BamHI fragment. After XbaI linker addition, T antigen cDNA was ligated in the unique XbaI site of pRC/CMV (Invitrogen) to form pRC/CMV-T. In this vector, T antigen cDNA is transcriptionally controlled by the cytomegalovirus (CMV) immediate-early promoter. pRC/CMV contains an SV40 DNA origin; pRC/CMV-T therefore contains a complete SV40 replicon.

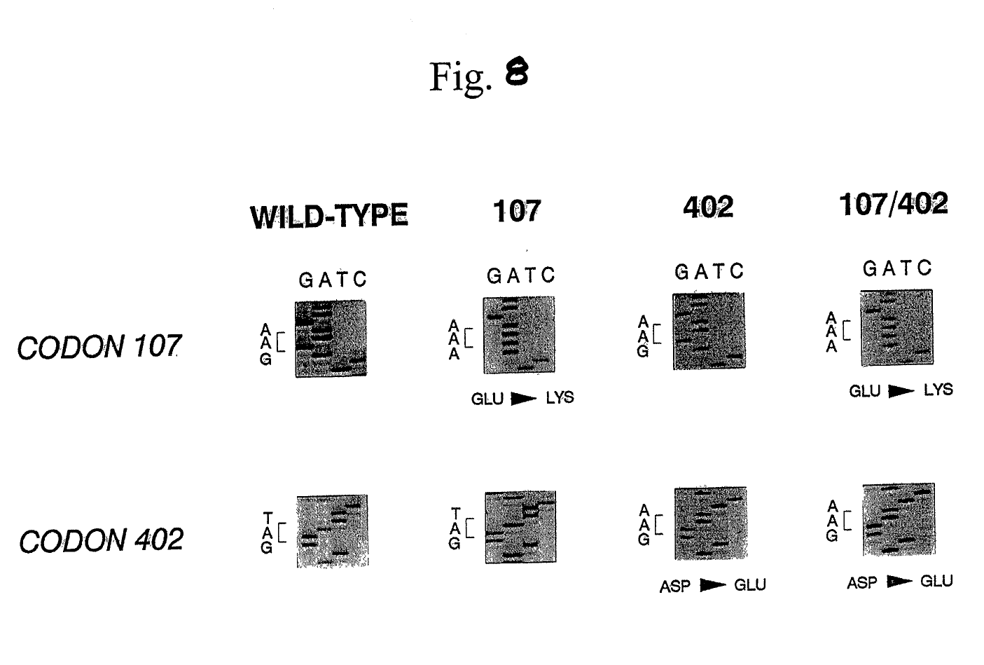

In a similar fashion, pRC/CMV.107-T was constructed from pSG5-K1, which encodes a mutant T antigen substituting lysine for glutamic acid at codon 107 (Kalderon and Smith, 1984). pRC/CMV.402-T and pRC/CMV.107/402-T were constructed by substituting a 1067 base pair HpaI C-terminal fragment of T antigen from pRC/CMV-T and pRC/CMV.107-T, respectively, with the corresponding T antigen fragment from a mutant SV40 virus clone that encodes a point mutation which substitutes glutamic acid for aspartic acid at codon 402 (clone 402DE) (Lin and Simmons, 1991). These point mutations are shown schematically in FIG. 8.

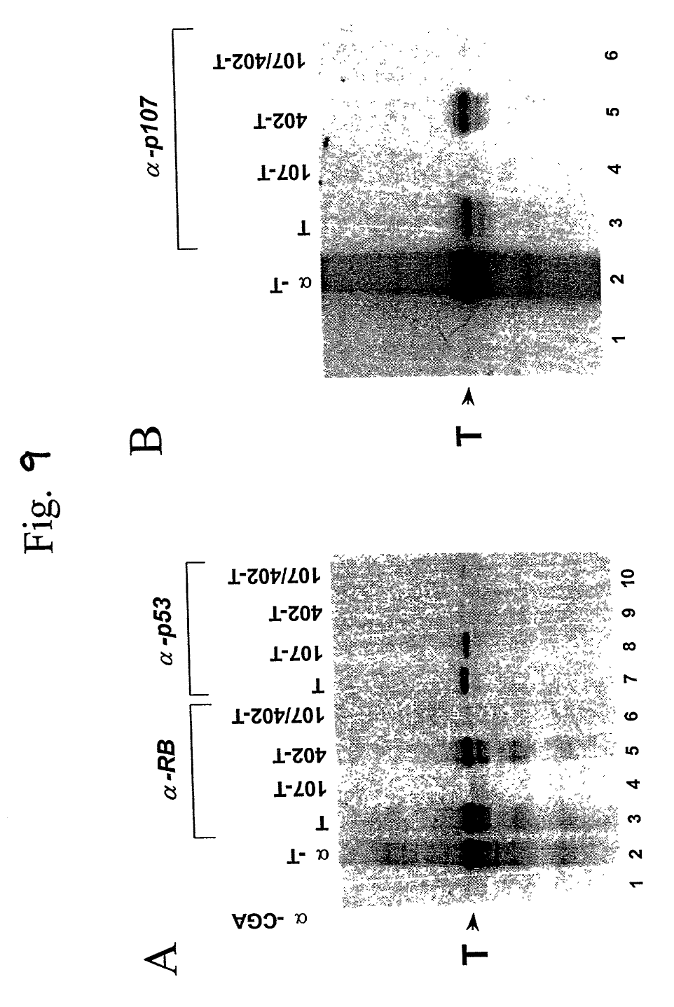

DNA sequence analysis confirmed in-frame ligation of the HpaI fragment, and also verified presence or absence of point mutations in codons 107 and 402 for each plasmid construct (FIG. 9).

### EXAMPLE 8

107/402-T Antigen does not Bind to Wild-Type RB, p107, and p53 Proteins

The biochemical correlate of SV40 large T antigen-mediated induction of tumorigenicity is complex formation with p53, RB, and possibly RB-related proteins such as p107 (Linzer and Levine, 1979; DeCaprio et al., 1988; Ewen et al., 1991; Claudio et al., 1994). To evaluate directly the ability of 107/402-T to bind to wild-type RB, p107, and p53, in vitro translated wild-type and mutant T antigens were added to extracts from CV-1 cells in which human RB, p107, or p53 were transiently expressed at high levels.

Wild-type and mutant T antigens were translated in vitro in the presence of ³⁵S-methionine, using a reticulocyte lysate system as described by the manufacturer (Promega). Labeled T antigen (2×10⁵ dpm) was added to extracts from CV-1 cells transiently expressing human RB, p107, or p53 at high levels. CV-1 cells were infected with a vaccinia virus vector encoding T7 RNA polymerase. One hour later cells were transfected with derivatives of the pTM1 plasmid (Moss et al., 1990) containing a T7 polymerase site immediately upstream of either human RB, p107, or p53 cDNA.

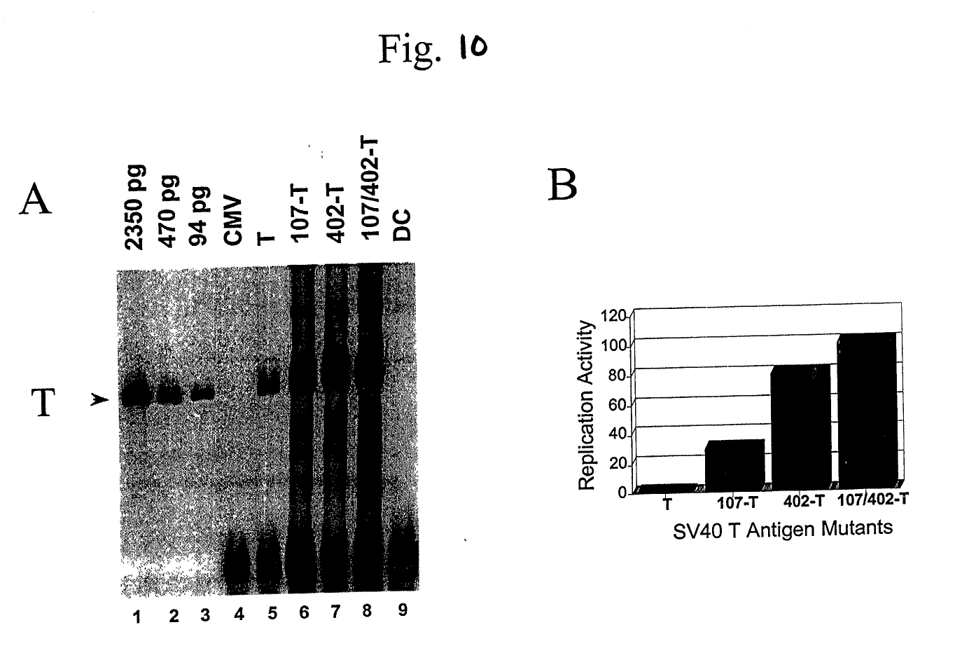

Approximately eighteen hours later, cells were harvested using a lysis buffer as described in Cooper et al. (1994). Immunoprecipitation analysis was performed using monoclonal antibodies to RB (clones G3-245, Pharmingen), p107 (clone SD9, Oncogene Science), and p53 (clone 1801, Oncogene Science), as described in DeCaprio et al., 1988. Band intensities were scanned using a phosphorimager to quantitate binding interactions. The results of these experiments are shown in FIGS. 10A and 10B.

As shown in Table 3, little or no binding of 107/402-T was detected in these experiments, demonstrating that 107/402-T does not bind significantly to either RB, p1 07, or p53.

TABLE 3
Binding of wild-type and mutant SV40 large T antigens to RB p107,
and p53 tumor suppressor gene products

| Tumor suppressor | Observed signal compared to T | | | |
|---|---|---|---|---|
| gene product | T, % | 107-T, % | 402-T, % | 107/402-T, % |
| RB | 100 | 0.03 | 67 | 0.07 |
| p107 | 100 | 0 | 79 | 0 |
| p53 | 100 | 36.2 | 0 | 0 |

### EXAMPLE 9

107/402-T is Replication-Competent and is a More Effective Replication Activator than Wild-Type Large T Antigen

The replication activities of wild-type and mutant SV40 large T antigens were evaluated in a panel of human cell lines, including HT-1376 (bladder carcinoma), 5637 (bladder carcinoma), MCF-7 (breast carcinoma), SW480 (colon cancer), Hs68 (fibroblast), HepG2 (hepatoma), and RAJI (lymphoma).

Cells were transfected using either lipofectin (GIBCO) (Cooper and Miron, 1993), calcium phosphate DNA precipitation (Graham and Van der Eb, 1973), or electroporation. Specific transfection conditions were optimized to achieve a transfection efficiency of at least 1% while minimizing cell toxicity. The day after gene transfer, cell cultures were split to maintain log phase growth for the duration of the experiment.

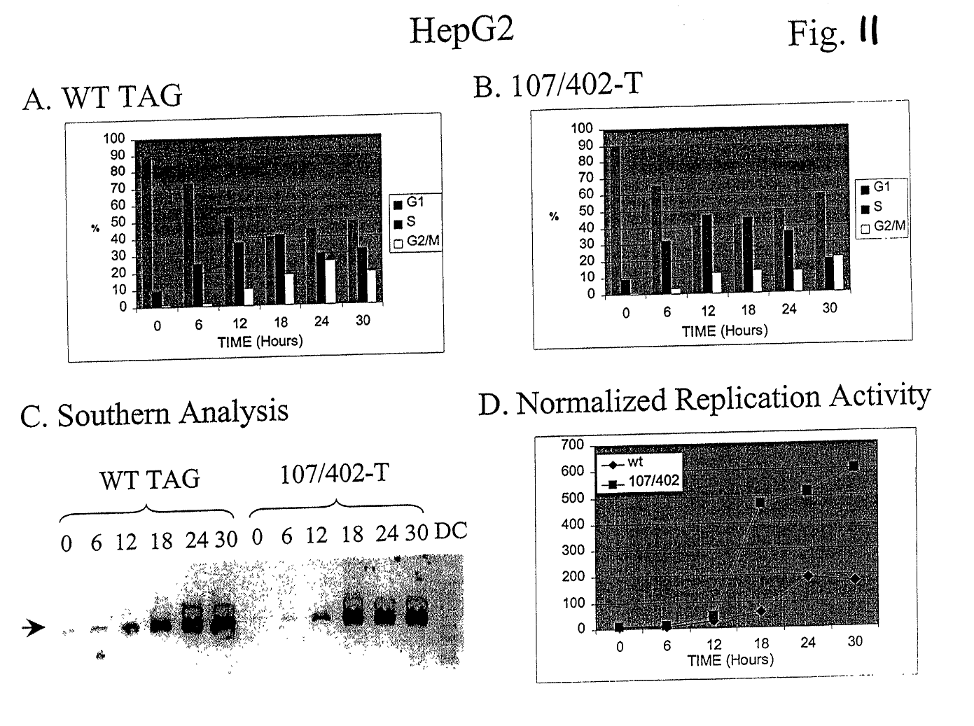

DNA harvested from transient transfectants was evaluated for the presence of extrachromosomal plasmid replication by resistance to DpnI digestion, as described in Cooper and Miron (1993). As shown in FIG. 11A, significant replication activity was observed in human cells. In HepG2 cells, for example, a copy number of approximately 25,000 per cell was noted by two days post-gene transfer, and copy numbers ranging from 80 to 100,000 were observed in other human cell types (Cooper et al. 1997). Furthermore, in the HepG2 cell line the replication activating ability of 107/402-T was increased over that of wild-type SV40 large T antigen by a factor of one hundred (FIGS. 11A and 11B).

### EXAMPLE 10

107/402-T has Enhanced Replication Activity Compared to Wild-Type T Antigen during S-Phase of the Cell Cycle

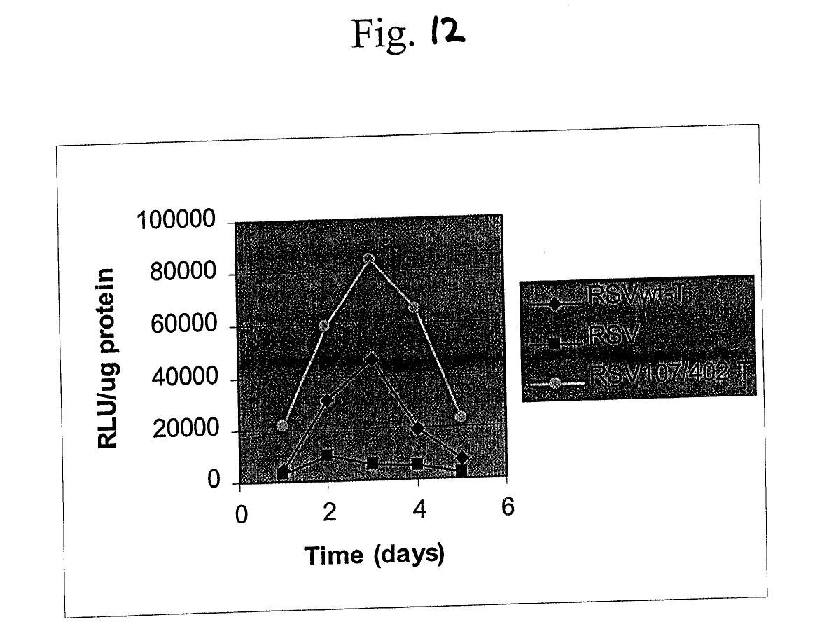

As described in Example 9, the copy number of pRC/CMV.107/402-T in HepG2 human hepatoma cells was 100-fold higher than pRC/CMV.T at 2 days post gene transfer. To further investigate the mechanism underlying this difference in episomal copy number, the cell cycle dependence of replication activity was evaluated. HepG2 cells were transfected with pRC/CMV.107/402-T or pRC/CMV.T. Twenty-four hours later, cells in early G1 of the cell cycle were isolated by centrifugal elutriation. The initial population of G1-enriched cells (time 0) and cells 6, 12, 18, 24, and 30 hours after replating were assayed for cell cycle analysis (FACS analysis of propidium iodide-stained cells, FIGS. 12A and 12B) and episomal copy number (Southern blot analysis, FIG. 12C). Log-phase growth conditions were maintained during replating. The band intensities in FIG. 12C were normalized for transient transfection efficiency by FACS analysis of T antigen expression. The normalized replication activity is presented in FIG. 12D.

The cell cycle analysis demonstrated that the peak time period for traversal of S phase was 12-18 hours post-replating. By 30 hours post-replating, cells transfected with pRC/CMV.107/402-T had a 3.4-fold increase in copy number compared to pRC/CMV.T. The increase in replication activity appeared to be largely restricted to S-phase of the cell cycle and accounts for the enhanced replication activity of 107/402-T in comparison to wild-type T antigen.

### EXAMPLE 11

107/402-T Significantly Enhances Gene Expression Compared to Wild-Type Antigen

To evaluate replication reporter transgene expression mediated by 107/402-T or wild-type T antigen, HepG2 hepatoma cells were co-transfected with pCMVSEAP (CMV immediately-early promoter transcribing secreted alkaline phosphatase) and either pRSVwt-T, pRSV.107/402-T, or pRSV (no insert). These RSV expression vectors lack an SV40 DNA origin and hence will not replicate in transiently transfected cells; the duration of T antigen expression will therefore be limited. In contrast, pCMVSEAP contains an SV40 DNA origin and will replicate extrachromosomally in cells co-expressing T antigen. HepG2 cells in 100 mm dishes were cotransfected with 10 ng of pCMVSEAP and 14 μg the RSV-based vectors.

On day one, samples of medium from the cells were saved and the cells were trypsinized and replated. At each 24 hour interval, media was harvested and cell extracts were prepared to calculate the total amount of protein per well. Alkaline phosphatase activity was measured in media using a commercial chemiluminescent assay (Tropix).

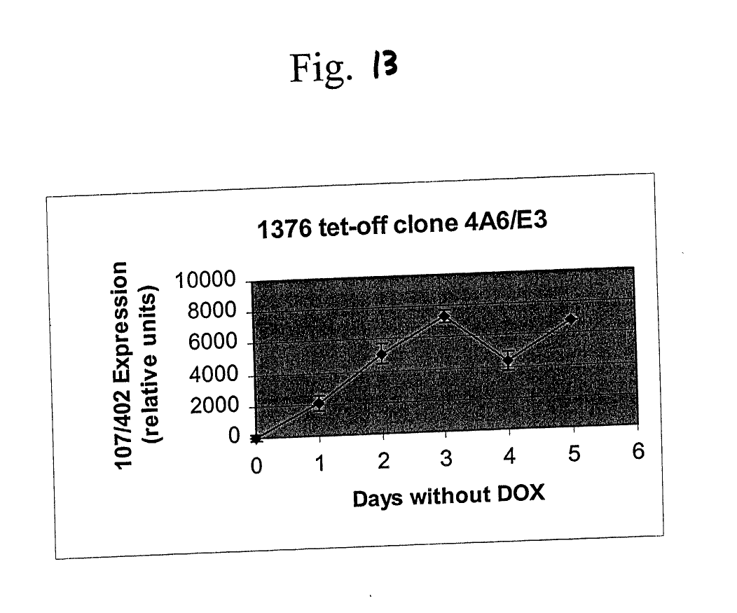

Data are presented in FIG. 13 as relative light units per μg of protein per 24 hours. A two- to five-fold improvement in alkaline phosphatase activity was observed in the 107/402-T co-transfectants compared to the wild-type T antigen co-transfectants. The level of alkaline phosphatase activity in the 107/402-T co-transfectants was greater than an order of magnitude higher than the pRSV co-transfectants (non-replicating standard expression vector control), emphasizing the importance of this replicating expression system for producing high levels of recombinant protein.

### EXAMPLE 12

Gene-Modified Cells can be Prepared to Express 107/402-T under Transcriptional Control of the Tetracycline-Controlled Gene Switch

To prepare a human cell line in which expression of 107/402-T would be under control of doxycycline, HT-1376 human bladder carcinoma cells were sequentially transfected with three plasmid constructs: (a) pTET-OFF, which encodes the tetracycline-controlled transcriptional transactivator (tTA) under control of the CMV immediate-early promoter and the neomycin resistance gene under control of the SV40 early promoter, (b) pTRE.107/402-T, which encodes 107/402-T under control of the CMV minimal promoter and contains the tetracycline operon (binding site of tTA just upstream of the CMV minimal promoter), and (c) pCMVhygro, which encodes the hygromycin resistance gene under control of the CMV promoter. HT-1376 cells were first transfected with pTET-OFF, and neomycin resistant clones of stable transfectants were characterized by transiently transfecting clones with pTRE.luciferase in the presence or absence of doxycycline. Clones which yielded significant luciferase activity only in the absence of doxycycline (but no detectable luciferase activity in the presence of doxycycline) were then co-transfected with pTRE.107/402-T and pCMVhygro. Again, single cell clones of stable transfectants were screened for high basal levels of 107/402-T and complete turn-off of 107/402-T expression in the presence of doxycycline.

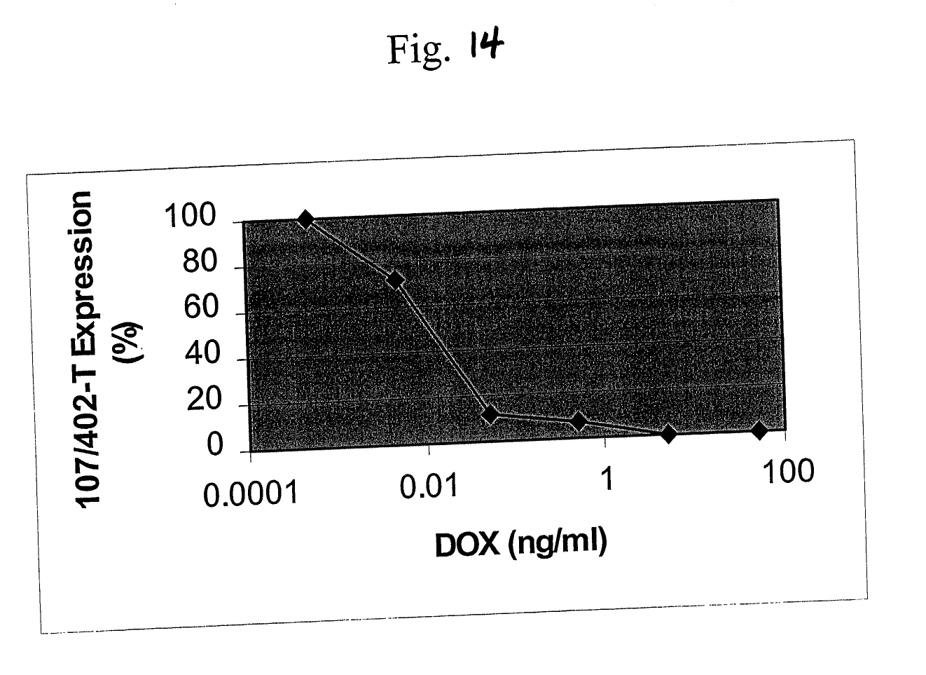

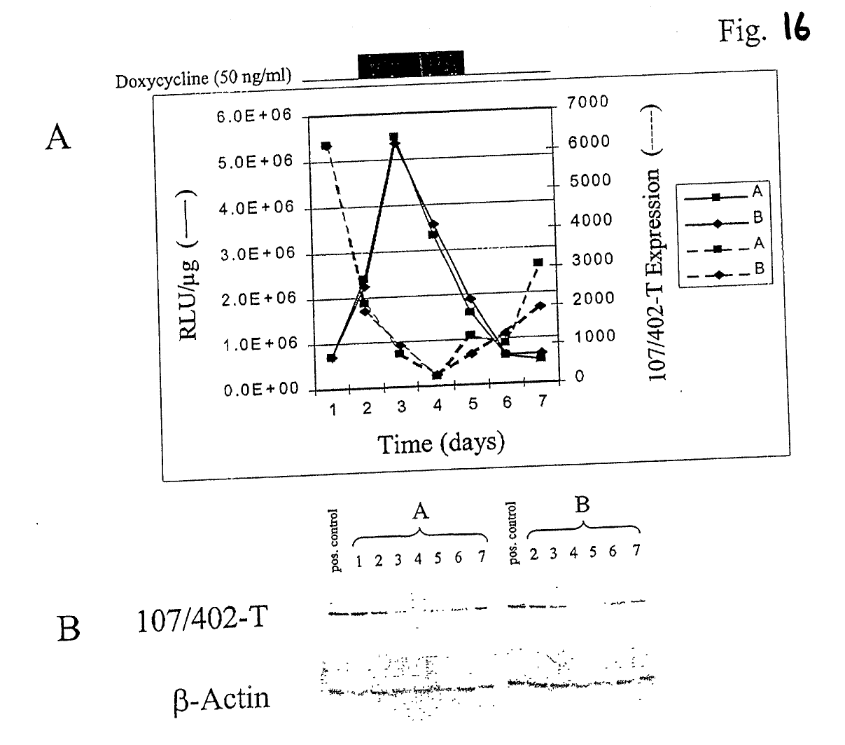

An example of an HT-1376 tet-off clone that demonstrates precise control of 107/402-T expression is shown in FIG. 14. FIG. 14 shows the time course of induction of 107/402-T expression upon washout of saturating amounts of doxycycline (3 ng/ml). Steady-state levels of 107/402-T are achieved by 3 days. The doxycycline concentration-dependence of 107/402-T expression is presented in FIG. 15. The half-maximal inhibitory concentration of doxycycline is approximately 0.01 ng/ml. The half-life of 107/402-T expression after addition of 3 ng/ml doxycycline is presented in FIG. 16. The observed decrease of 107/402-T expression yields a half-life of 22.7 hours in this cell line.

The data in FIGS. 14-16 permit design of a cyclic regimen of doxycyline that will fluctuate levels of 107/402-T expression about a predetermined level of 107/402-T expression. Such a cyclic profile of 107/402-T expression will, in turn, generate sustained and elevated levels of transgene expression derived from a replicating reporter plasmid encoding the SV40 DNA origin.

### EXAMPLE 13

Cyclic Regimens of Doxycycline can be Used to Control Transgene Expression in the Gene-Modified Cells of Example 12

The HT-1376 clone described in Example 12 (clone 4A6/E3) was transfected with pCMVSEAP, an expression plasmid in which the CMV immediate-early promoter regulates transcription of a secreted alkaline phosphatase reporter gene. pCMVSEAP contains the SV40 DNA origin and hence will replicate extrachromosomally in the presence of 107/402-T antigen. In this experiment, cells were incubated without doxycycline for 4 days to produce maximal levels of 107/402-T antigen expression. Duplicate dishes of cells (A, B) were then transfected with pCMVSEAP (day 0) and replated in a series of 60 mm wells for analysis of alkaline phosphatase expression at a series of time points. Doxycycline (50 ng/ml) was added back to the cells between days 2-5 to block production of 107/402-T antigen. Media was changed every 24 hours to determine daily alkaline phosphatase activity.

To measure secreted alkaline phosphatase activity, 25 μl of media were assayed using a chemiluminescent assay as described by the manufacturer (Tropix, Inc.). The light units per well were then calculated, and cells were harvested for protein determination. Activity is expressed as relative light units of alkaline phosphatase activity per μg of protein per 24 hours.

As shown in FIG. 17A, alkaline phosphatase activity peaks on day 3 and then declines. In FIG. 17B, the protein extracts were evaluated for 107/402-T expression by Western blot analysis. The same blot was reprobed for β-actin to ensure equal loading of extracts per well. Normalized levels of 107/402-T antigen expression are plotted in FIG. 17A and demonstrate that levels of 107/402-T antigen can be cycled by appropriate exposure of the cells to doxycycline.

These data demonstrate the ability to cycle levels of transgene expression using the method of the invention. Furthermore, levels of transgene expression can be optimized for a given application by simply altering the regimen of doxycycline exposure to yield appropriate levels of episomal amplification. This modular and flexible system permits optimization of expression for a given transgene based on potential toxicities of the transgene to the host production cell as well as the inherent synthetic capabilities of the producer cell.

### REFERENCES

Boshart et al.(1985)

Caras and Weddell (1989)

Chenetal. (1990) J. Virol. 64, 3350-57

Chen et al. (1992) Oncogene 7, 1167-75

Christian et al. (1987) Cancer Res. 47, 6066-73

Claudio et al. (1994) Cancer Res. 54, 5556-60

Cooper et al. (1994) Oncol. Res. 6, 569-79

Cooper et al. (1997) Proc. Natl. Acad. Sci. U.S.A. 94, 6450-55

Cooper and Miron (1993) Human Gene Ther. 4, 557-66

DeLort and Capecchi (1996) Human Gene Therapy 7, 809-20

DeCaprio et al. (1988) Cell 54, 275-83

Deyerle et al. (1989) J. Virol. 63, 356-65

Ewen et al. (1991) Cell 66, 1155-64

Fiers et al. (1978) Nature 273, 113-20

Freshney, ed. (1986) ANIMAL CELL CULTURE

Gerard and Gluzman (1985) Mol. Cell. Biol. 5, 3231-40

Glover, ed. (1985) DNA CLONING: A PRACTICAL APPROACH, vols. 1 and 2

Gossen and Bujard (1992) Proc. Natl. Acad. Sci. U.S.A. 89, 5547-51

Gossen (1995) Science 268, 1766-69

Guo et al. (1996) Gene Ther. 3, 802-10

Hanahan et al. (1989) Science 246, 1265-75

Huber et al. (1991) Proc. Natl. Acad. Sci. U.S.A. 88, 8039-43

Kalderon and Smith (1984) Virol. 139, 109-37

Lin and Simmons (1991) J. Virol. 65, 2066-72

Linzer and Levin (1979) Cell 17, 43-52

Michalovitz et al. (1987) J. Virol. 61, 2648-54

Moss et al. (1990) Nature 348, 91-92

Nordeen (1988) BioTechniques 6, 454-48

Osali et al. (1994) Cancer Res. 54, 5258-61

Perbal (1984) A PRACTICAL GUIDE TO MOLECULAR CLONING

Reddy et al. (1978) Science 200 494-502

Rivera et al. (1996) Nature Med. 2, 1028-32

Roberts et al. (1986) Cell 46, 741-52

Sambrook et al. (1989) MOLECULAR CLONING: A LABORATORY MANUAL

Shin et al. (1975) Proc. Natl. Acad. Sci. U.S.A. 72, 4435-39

Subramanian et al. (1977) J. Biol. Chem. 252, 355-67

Van Heuverswyn et al. (1978) Eur. J. Biochem. 100, 51-60

Vegeto et al. (1992) Cell 69, 703-13

Yang et al. (1979) Science 206, 456-61

Walter and Blobel (1982) Nature 299, 691-98

Wang et al. (1997) Gene Ther. 4, 432-41

All patents and patent applications cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

1. A kit, comprising:
(a) a DNA molecule comprising:
(1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen; and
(2) a first promoter which controls expression of the mutant form of the papovavirus large T antigen; and
(b) a first episome comprising:
(1) the papovavirus origin of replication; and
(2) a coding sequence for a protein or a site for inserting the coding sequence for the protein.

2. The kit of claim 1 wherein the first episome comprises the DNA molecule.

3. The kit of claim 1 wherein the papovavirus large T antigen is an SV40 large T antigen.

4. The kit of claim 1 wherein the papovavirus large T antigen is a BK large T antigen.

5. The kit of claim 1 wherein the papovavirus origin of replication is an SV40 origin of replication.

6. The kit of claim 1 wherein the papovavirus origin of replication is a BK origin of replication.

7. The kit of claim 1 wherein the papovavirus large T antigen is an SV40 large T antigen and wherein the papovavirus origin of replication is an SV40 origin of replication.

8. The kit of claim 1 wherein the papovavirus large T antigen is a BK large T antigen and wherein the papovavirus origin of replication is a BK origin of replication.

9. The kit of claim 1 wherein the mutant form of papovavirus large T antigen is an SV40 large T antigen in which residue 107 is lysine and residue 402 is glutamic acid.

10. The kit of claim 1 wherein the first promoter is an inducible promoter.

11. The kit of claim 1 wherein the first promoter is a metallothionene promoter.

12. The kit of claim 1 wherein the first promoter is a promoter for a developmentally-controlled gene.

13. The kit of claim 1 wherein the first promoter is a promoter for a tissue-specific gene.

14. The kit of claim 1 wherein the first promoter is a promoter for a breast-specific gene.

15. The kit of claim 1 wherein the first promoter is under hormonal control.

16. The kit of claim 1 wherein the protein is a cytokine.

17. The kit of claim 1 wherein the protein is an interleukin.

18. The kit of claim 1 wherein the protein confers susceptibility to a chemotherapeutic agent.

19. The kit of claim 1 wherein the protein is Herpes simplex thymidine kinase.

20. The kit of claim 1 wherein the protein is cytosine deaminase.

21. The kit of claim 1 wherein the protein is capable of inducing apoptosis.

22. The kit of claim 1 wherein the protein is an anti-oncogenic protein.

23. The kit of claim 1 wherein the protein is p53.

24. The kit of claim 1 wherein the coding sequence for the protein is in the antisense orientation.

25. The kit of claim 24 wherein the protein is an oncogenic protein.

26. The kit of claim 1 further comprising a mammalian cell which can be transfected with the DNA molecule and the first episome.

27. The kit of claim 26 wherein the first episome comprises the DNA molecule.

28. The kit of claim 1 further comprising a mammalian cell which can be transfected with the first episome, wherein the DNA molecule is integrated into the genome of the cell.

29. The kit of claim 1 further comprising a mammalian cell which comprises the DNA molecule and the first episome.

30. The kit of claim 29 wherein the DNA molecule is integrated into the genome of the mammalian cell.

31. The kit of claim 29 wherein the DNA molecule is a second episome.

32. The kit of claim 29 wherein the first episome comprises the DNA molecule.

33. The kit of claim 26, 27, 28, 29, 30, 31, or 32 wherein the mammalian cell is a human cell.

34. The kit of claim 26, 27, 28, 29, 30, 31, or 32 wherein the mammalian cell is a bladder cell.

35. The kit of claim 26, 27, 28, 29, 30, 31, or 32 wherein the mammalian cell is a breast cell.

36. The kit of claim 26, 27, 28, 29, 30, 31, or 32 wherein the mammalian cell is a peripheral blood monocyte.

37. The kit of claim 26, 27, 28, 29, 30, 31, or 32 wherein the mammalian cell is a stem cell.

38. The kit of claim 26, 27, 28, 29, 30, 31, or 32 wherein the mammalian cell is a tumor cell.

39. The kit of claim 26, 27, 28, 29, 30, 31, or 32 wherein the mammalian cell is a non-tumor cell.

40. The kit of claim 1, 26, 27, 28, 29, 30, 31, or 32 wherein the first episome further comprises a second promoter which controls expression of the protein.

41. The kit of claim 40 wherein the second promoter is an inducible promoter.

42. The kit of claim 40 wherein the second promoter is a metallothionene promoter.

43. The kit of claim 40 wherein the second promoter is a promoter for a developmentally-controlled gene.

44. The kit of claim 40 wherein the second promoter is a promoter for a tissue-specific gene.

45. The kit of claim 40 wherein the second promoter is a promoter for a breast-specific gene.

46. The kit of claim 40 wherein the second promoter is under hormonal control.

47. A kit, comprising:
(a) a mammalian cell;
(b) a DNA molecule comprising:
(1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen, wherein the DNA molecule is integrated into the genome of the mammalian cell; and
(2) a first promoter which controls expression of the mutant form of the papovavirus large T antigen; and
(c) a first episome comprising:
(1) the papovavirus origin of replication; and
(2) a coding sequence for a protein or a site for inserting the coding sequence for the protein; and
(3) a second promoter for controlling expression of the coding sequence for the protein.

48. A kit, comprising:
(a) a mammalian cell; and
(b) an episome comprising:
(1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen;
(2) a promoter which controls expression of the mutant form of the papovavirus large T antigen;
(3) the papovavirus origin of replication; and
(4) a coding sequence for a protein or a site for inserting the coding sequence for the protein.

49. A mammalian cell comprising:
(a) a DNA molecule comprising:
(1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen; and
(2) a first promoter which controls expression of the mutant form of the papovavirus large T antigen; and
(b) a first episome comprising:
(1) a coding sequence for a protein or a site for inserting the coding sequence for the protein; and
(2) the papovavirus origin of replication.

50. The mammalian cell of claim 49 wherein the DNA molecule is integrated into the genome of the mammalian cell.

51. The mammalian cell of claim 49 wherein the DNA molecule is a second episome.

52. The mammalian cell of claim 49 wherein the first episome comprises the DNA molecule.

53. The mammalian cell of claim 49 wherein the papovavirus large T antigen is an SV40 large T antigen.

54. The mammalian cell of claim 49 wherein the papovavirus large T antigen is a BK large T antigen.

55. The mammalian cell of claim 49 wherein the papovavirus origin of replication is an SV40 origin of replication.

56. The mammalian cell of claim 49 wherein the papovavirus origin of replication is a BK origin of replication.

57. The mammalian cell of claim 49 wherein the papovavirus large T antigen is an SV40 large T antigen and wherein the papovavirus origin of replication is an SV40 origin of replication.

58. The mammalian cell of claim 49 wherein the papovavirus large T antigen is a BK large T antigen and wherein the papovavirus origin of replication is a BK origin of replication.

59. The mammalian cell of claim 49 wherein the mutant form of the papovavirus large T antigen is an SV40 large T antigen in which residue 107 is lysine and residue 402 is glutamic acid.

60. The mammalian cell of claim 49 wherein the first promoter is an inducible promoter.

61. The mammalian cell of claim 49 wherein the first promoter is a metallothionene promoter.

62. The mammalian cell of claim 49 wherein the first promoter is a promoter for a developmentally-controlled gene.

63. The mammalian cell of claim 49 wherein the first promoter is a promoter for a tissue-specific gene.

64. The mammalian cell of claim 49 wherein the first promoter is a promoter for a breast-specific gene.

65. The mammalian cell of claim 49 wherein the first promoter is under hormonal control.

66. The mammalian cell of claim 49 wherein the protein is a cytokine.

67. The mammalian cell of claim 49 wherein the protein is an interleukin.

68. The mammalian cell of claim 49 wherein the confers susceptibility to a chemotherapeutic agent.

69. The mammalian cell of claim 49 wherein the protein is Herpes simplex thymidine kinase.

70. The mammalian cell of claim 49 wherein the protein is cytosine deaminase.

71. The mammalian cell of claim 49 wherein the protein is capable of inducing apoptosis.

72. The mammalian cell of claim 49 wherein the protein is an anti-oncogenic protein.

73. The mammalian cell of claim 49 wherein the protein is p53.

74. The mammalian cell of claim 49 wherein the coding sequence for the protein is in the antisense orientation.

75. The mammalian cell of claim 74 wherein the protein is an oncogenic protein.

76. The mammalian cell of claim 49, 50, 51 or 52 which is a human cell.

77. The mammalian cell of claim 49, 50, 51 or 52 which is a bladder cell.

78. The mammalian cell of claim 49, 50, 51 or 52 which is a breast cell.

79. The mammalian cell of claim 49, 50, 51 or 52 which is a peripheral blood monocyte.

80. The mammalian cell of claim 49, 50, 51 or 52 which is a stem cell.

81. The mammalian cell of claim 49, 50, 51 or 52 which is a tumor cell.

82. The mammalian cell of claim 49, 50, 51 or 52 which is a non-tumor cell.

83. The mammalian cell of claim 49, 50, 51 or 52 wherein the first episome further comprises a second promoter which controls expression of the coding sequence for the protein.

84. The mammalian cell of claim 83 wherein the second promoter is an inducible promoter.

85. The mammalian cell of claim 83 wherein the second promoter is a metallothionene promoter.

86. The mammalian cell of claim 83 wherein the second promoter is a promoter for a developmentally-controlled gene.

87. The mammalian cell of claim 83 wherein the second promoter is a promoter for a tissue-specific gene.

88. The mammalian cell of claim 83 wherein the second promoter is a promoter for a breast-specific gene.

89. The mammalian cell of claim 83 wherein the second promoter is under hormonal control.

90. A mammalian cell, comprising:
a DNA molecule which comprises:
(a) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen; and
(b) a promoter which controls expression of the mutant form of the papovavirus large T antigen.

91. The mammalian cell of claim 90 wherein the DNA molecule is integrated into the genome of the mammalian cell.

92. The mammalian cell of claim 90 wherein the DNA molecule is an episome.

93. The mammalian cell of claim 90 wherein the papovavirus large T antigen is an SV40 large T antigen.

94. The mammalian cell of claim 90 wherein the papovavirus large T antigen is a BK large T antigen.

95. The mammalian cell of claim 90 wherein the papovavirus origin of replication is an SV40 origin of replication.

96. The mammalian cell of claim 90 wherein the papovavirus origin of replication is a BK origin of replication.

97. The mammalian cell of claim 90 wherein the papovavirus large T antigen is an SV40 large T antigen and wherein the papovavirus origin of replication is an SV40 origin of replication.

98. The mammalian cell of claim 90 wherein the papovavirus large T antigen is a BK large T antigen and wherein the papovavirus origin of replication is a BK origin of replication.

99. The mammalian cell of claim 90 wherein the mutant form of the papovavirus large T antigen is an SV40 large T antigen in which residue 107 is lysine and residue 402 is glutamic acid.

100. The mammalian cell of claim 90 wherein the promoter is an inducible promoter.

101. The mammalian cell of claim 90 wherein the promoter is a metallothionene promoter.

102. The mammalian cell of claim 90 wherein the promoter is a promoter for a developmentally-controlled gene.

103. The mammalian cell of claim 90 wherein the promoter is a promoter for a tissue-specific gene.

104. The mammalian cell of claim 90 wherein the promoter is a promoter for a breast-specific gene.

105. The mammalian cell of claim 90 wherein the promoter is under hormonal control.

106. The mammalian cell of claim 90, 91, or 92 which is a human cell.

107. The mammalian cell of claim 90, 91, or 92 which is a bladder cell.

108. The mammalian cell of claim 90, 91, or 92 which is a breast cell.

109. The mammalian cell of claim 90, 91, or 92 which is a peripheral blood monocyte.

110. The mammalian cell of claim 90, 91, or 92 which is a stem cell.

111. The mammalian cell of claim 90, 91, or 92 which is a tumor cell.

112. The mammalian cell of claim 90, 91, or 92 which is a non-tumor cell.

113. A method, comprising the step of:
culturing a mammalian cell which comprises:
(a) a DNA molecule comprising:
(1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen; and
(2) a first promoter which controls expression of the mutant form of the papovavirus large T antigen; and
(b) a first episome comprising:
(1) a coding sequence for a protein; and
(2) the papovavirus origin of replication, wherein the step of culturing is carried out under conditions suitable for expressing the protein.

114. The method of claim 113 wherein the DNA molecule is integrated into the genome of the mammalian cell.

115. The method of claim 113 wherein the DNA molecule is a second episome.

116. The method of claim 113 wherein the first episome comprises the DNA molecule.

117. The method of claim 113 wherein the papovavirus large T antigen is an SV40 large T antigen.

118. The method of claim 113 wherein the papovavirus large T antigen is a BK large T antigen.

119. The method of claim 113 wherein the papovavirus origin of replication is an SV40 origin of replication.

120. The method of claim 113 wherein the papovavirus origin of replication is a BK origin of replication.

121. The method of claim 113 wherein the papovavirus large T antigen is an SV40 large T antigen and wherein the papovavirus origin of replication is an SV40 origin of replication.

122. The method of claim 113 wherein the papovavirus large T antigen is a BK large T antigen and wherein the papovavirus origin of replication is a BK origin of replication.

123. The method of claim 113 wherein the mutant form of papovavirus large T antigen is an SV40 large T antigen in which residue 107 is lysine and residue 402 is glutamic acid.

124. The method of claim 113 wherein the first promoter is an inducible promoter.

125. The method of claim 113 wherein the first promoter is a metallothionene promoter.

126. The method of claim 113 wherein the first promoter is a promoter for a developmentally-controlled gene.

127. The method of claim 113 wherein the first promoter is a promoter for a tissue-specific gene.

128. The method of claim 113 wherein the first promoter is a promoter for a breast-specific gene.

129. The method of claim 113 wherein the first promoter is under hormonal control.

130. The method of claim 113 wherein protein is a cytokine.

131. The method of claim 113 wherein the protein is an interleukin.

132. The method of claim 113 wherein the protein confers susceptibility to a chemotherapeutic agent.

133. The method of claim 113 wherein the protein is Herpes simplex thymidine kinase.

134. The method of claim 113 wherein the protein is cytosine deaminase.

135. The method of claim 113 wherein the protein is capable of inducing apoptosis.

136. The method of claim 113 wherein the protein is an anti-oncogenic protein.

137. The method of claim 113 wherein protein is p53.

138. The method of claim 113 wherein the coding sequence for the protein is in the antisense orientation.

139. The method of claim 138 wherein the protein is an oncogenic protein.

140. The method of claim 113, 113, 114, 115, or 116 wherein the mammalian cell is a human cell.

141. The method of claim 113, 113, 114, 115, or 116 wherein the mammalian cell is a bladder cell.

142. The method of claim 113, 113, 114, 115, or 116 wherein the mammalian cell is a breast cell.

143. The method of claim 113, 113, 114, 115, or 116 wherein the mammalian cell is a peripheral blood monocyte.

144. The method of claim 113, 114, 115, or 116 wherein the mammalian cell is a stem cell.

145. The method of claim 113, 113, 114, 115, or 116 wherein the mammalian cell is a tumor cell.

146. The method of claim 113, 113, 114, 115, or 116 wherein the mammalian cell is a non-tumor cell.

147. The method of claim 113, 113, 114, 115, or 116 wherein the first episome further comprises a second promoter which controls expression of the foreign gene.

148. The method of claim 147 wherein the second promoter is an inducible promoter.

149. The method of claim 147 wherein the second promoter is a metallothionene promoter.

150. The method of claim 147 wherein the second promoter is a promoter for a developmentally-controlled gene.

151. The method of claim 147 wherein the second promoter is a promoter for a tissue-specific gene.

152. The method of claim 147 wherein the second promoter is a promoter for a breast-specific gene.

153. The method of claim 147 wherein the second promoter is under hormonal control.

154. A method comprising the step of:
transfecting a mammalian cell with a first episome comprising (a) a coding sequence for a protein and (b) a papovavirus origin of replication, wherein the mammalian cell comprises a DNA molecule which comprises:
(a) a coding sequence for a mutant form of a papovavirus large T antigen which contains a replication-competent binding site for the papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen; and
(b) a first promoter which controls expression of the mutant form of the papovavirus large T antigen.

155. The method of claim 154 wherein the DNA molecule is integrated into the genome of the mammalian cell.

156. The method of claim 154 wherein the DNA molecule is a second episome.

157. The method of claim 154 wherein the papovavirus large T antigen is an SV40 large T antigen.

158. The method of claim 154 wherein the papovavirus large T antigen is a BK large T antigen.

159. The method of claim 154 wherein the papovavirus origin of replication is an SV40 origin of replication.

160. The method of claim 154 wherein the papovavirus origin of replication is a BK origin of replication.

161. The method of claim 154 wherein the papovavirus large T antigen is an SV40 large T antigen and wherein the papovavirus origin of replication is an SV40 origin of replication.

162. The method of claim 154 wherein the papovavirus large T antigen is a BK large T antigen and wherein the papovavirus origin of replication is a BK origin of replication.

163. The method of claim 154 wherein the mutant form of papovavirus large T antigen is an SV40 large T antigen in which residue 107 is lysine and residue 402 is glutamic acid.

164. The method of claim 154 wherein the first promoter is an inducible promoter.

165. The method of claim 154 wherein the first promoter is a metallothionene promoter.

166. The method of claim 154 wherein the first promoter is a promoter for a developmentally-controlled gene.

167. The method of claim 154 wherein the first promoter is a promoter for a tissue-specific gene.

168. The method of claim 154 wherein the first promoter is a promoter for a breast-specific gene.

169. The method of claim 154 wherein the first promoter is under hormonal control.

170. The method of claim 154 wherein protein is a cytokine.

171. The method of claim 154 wherein the protein is an interleukin.

172. The method of claim 154 wherein the protein confers susceptibility to a chemotherapeutic agent.

173. The method of claim 154 wherein the protein is Herpes simplex thymidine kinase.

174. The method of claim 154 wherein the protein is cytosine deaminase.

175. The method of claim 154 wherein the protein is capable of inducing apoptosis.

176. The method of claim 154 wherein the protein is an anti-oncogenic protein.

177. The method of claim 154 wherein the protein is p53.

178. The method of claim 157 wherein the coding sequence for the protein is in the antisense orientation.

179. The method of claim 178 wherein the protein is an oncogenic protein.

180. The method of claim 154, 155, or 156 wherein the mammalian cell is a human cell.

181. The method of claim 154, 155, or 156 wherein the mammalian cell is a bladder cell.

182. The method of claim 154, 155, or 156 wherein the mammalian cell is a breast cell.

183. The method of claim 154, 155, or 156 wherein the mammalian cell is a peripheral blood monocyte.

184. The method of claim 154, 155, or 156 wherein the mammalian cell is a stem cell.

185. The method of claim 154, 155, or 156 wherein the mammalian cell is a tumor cell.

186. The method of claim 154, 155, or 156 wherein the mammalian cell is a non-tumor cell.

187. The method of claim 154, 155, or 156 wherein the first episome further comprises a second promoter which controls expression of the foreign gene.

188. The method of claim 187 wherein the second promoter is an inducible promoter.

189. The method of claim 187 wherein the second promoter is a metallothionene promoter.

190. The method of claim 187 wherein the second promoter is a promoter for a developmentally-controlled gene.

191. The method of claim 187 wherein the second promoter is a promoter for a tissue-specific gene.

192. The method of claim 187 wherein the second promoter is a promoter for a breast-specific gene.

193. The method of claim 187 wherein the second promoter is under hormonal control.

194. The method of claim 154 further comprising the step of culturing the mammalian cell under conditions suitable for expressing the coding sequence.

195. The kit of claim 24 wherein the protein controls cell growth.

196. The mammalian cell of claim 74 wherein the protein controls cell growth.

197. The method of claim 138 wherein the protein controls cell growth.

198. The method of claim 178 wherein the protein controls cell growth.

199. A kit, comprising:
(a) a mammalian cell;
(b) a first episome comprising:
(1) a coding sequence for a mutant form of a papovavirus large T antigen which contains a replication-competent binding site for a papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen; and
(2) a first promoter which controls expression of the mutant form of the papovavirus large T antigen; and
(c) a second episome comprising:
(1) the papovavirus origin of replication; and
(2) a coding sequence for a protein or a site for inserting the coding sequence for the protein; and
(3) a second promoter which controls expression of the coding sequence for the protein.

200. A method comprising the step of:
transfecting a mammalian cell with an episome comprising:
(a) a coding sequence for a protein;
(b) a papovavirus origin of replication;
(c) a coding sequence for a mutant form of a papovavirus large T antigen which contains a replication-competent binding site for the papovavirus origin of replication and which is negative for binding to a wild-type p53 gene product due to a mutation in a p53 binding domain of the large T antigen and which is negative for binding to a wild-type retinoblastoma tumor suppressor gene product due to a mutation in an RB binding domain of the large T antigen; and
(d) a promoter which controls expression of the mutant form of the papovavirus large T antigen.
